SEQUENCE LISTING (35 sequences)

SEQ ID NO: 1 - DNA, 20 bp, Bos taurus
ctcagctgcatacgccttcc

SEQ ID NO: 2 - DNA, 21 bp, Bos taurus
acaggtcattgtcagcactcc

SEQ ID NO: 3 - DNA, 39 bp, Bos taurus
atgcccatggaacatatgatgtggtggctacttgtaaca

SEQ ID NO: 4 - DNA, 40 bp, Bos taurus
gcatcccgggctagagctcctttttgtcttcggccatcaa

SEQ ID NO: 5 - protein, 399 aa, Artificial Sequence
  Bovine pregastric esterase cloned by IPG, translation product, with native leader and two amino acids added to form an ER retention signal sequence
MWWLLVTVCFIHMSGNAFCFLGKIAKNPEASMNVSQMISYWGYPSEMHKVITADGYILQVYRIPHGKNNANHLGQRPVVFLQHGLLGSATNWISNLPKNSLGFLLADAGYDVWLGNSRGNTWAQEHLYYSPDSPEFWAFSFDEMAEYDLPSTIDFILRRTGQKKLHYVGHSQGTTIGFIAFSTNPTLAEKIKVFYALAPVATVKYTKSLFNKLALIPHFLFKIIFGDKMFYPHTFLEQFLGVEMCSRETLDVLCKNALFAITGVDNKNFNMSRLDVYIAHNPAGTSVQNTLHWRQAVKSGKFQAFDWGAPYQNLMHYHQPTPPIYNLTAMNVPIAVWSADNDLLADPQDVDLLLSKLSNLIYHKEIPNYNHLDFIWAMDAPQEVYNEIVSLMAEDKKEL

SEQ ID NO: 6 - DNA, 31 bp, Caenorhabditis elegans
cagctgcatacgccgaaaatgtcaccactcc

SEQ ID NO: 7 - DNA, 21 bp, Caenorhabditis elegans
ttacgaaatagtatctggaag

SEQ ID NO: 8 - protein, 591 aa, Caenorhabditis elegans
MKSLLLLFLLFLHTLCENVTTPNSEEDTDMTATPSTITPLLSTAQNPSLPKTSKLPLALKTPVPTFPFSSLATSDWLSSMPTIQTLLPPPLPLTTLEVPENENFGFTSLAPLWTLPTQPPAWSPMLDSPIKPIQNSMFPTFPTMPTLPTLPTLAPFTFPTLPPPTTMKPLNITIDPEALMDVPEIITHWGYPVETHKVVTVDGYILTLHRIPHSKNETSKSASKTPKPVVFLQHGLLCTSSIWLLNLPRQSAGYIFADQGYDVWLGNMRGNTYSKEHTRMTSADRRFWKFSWEEMARYDLPAMINYALKTTKRQNLYYVGHSQGALTMFAKMSEDPEMSKKIRKFFAMAPVARMSHVKGLFQNLGQIYEQYNLVYQVFGDGEFLTNNIFTKLLTDIFCDQAVNNPLCENFIFAVSGPNSNQFNNSRIGIYLAHNPAGTSSRNILHFAQMVKKKRMSRFDHGKDLNLKIYGAPSPPEYDIRKINSSIYLFYSDFDWLANPKDVEGFLIPMLPSKTLKKATKLRDFNHNDFLWGMRARKEIYDKIINTIKLDQRRVKLQNSMERFFERQSRNPTSGLDEETMMRLRNETMNLD

SEQ ID NO: 9 - DNA, 23 bp, Lactococcus lactis
ccatggcagtaatcaatatcgaa

SEQ ID NO: 10 - DNA, 24 bp, Lactococcus lactis
tattaactcaatcgttcttcttgc

SEQ ID NO: 11 - protein, 258 aa, Lactococcus lactis
MAVINIEYYSEVLGMNRKVNVIYPESSKVEDFSNTEIPVLYLLHGMSGNENSWMIRSGIERLIRHTNLAIVMPSTDLGFYVNTTYGMNYFDAIALELPKVIHNFFPNLSTKKEKNFIAGLSMGGYGAYRLALGTDHFSYAASLSGVLTFDGMEENFKENPAYWGGIFGNWETFKGSDNEILALADRKNEERPKLYAWCGKQDFLFPGNEYAIAELKKKGFDVTYESSDGVHEWYYWTKKIESVLQWLPINYKQEERLS

SEQ ID NO: 12 - DNA, 21 bp, Arabidopsis thaliana
tcatgagtatctccggtgctg

SEQ ID NO: 13 - DNA, 24 bp, Arabidopsis thaliana
actagttcaaccttcgaggctgag

SEQ ID NO: 14 - protein, 252 aa, Arabidopsis thaliana
MSISGAAVGSGRNLRRAVEFGKTHVVRPKGKHQATIVWLHGLGDNGSSWSQLLETLPLPNIKWICPTAPSQPISLFGGFPSTAWFDVVDINEDGPDDMEGLDVAAAHVANLLSNEPADIKLGVGGFSMGAATSLYSATCFALGKYGNGNPYPINLSAIIGLSGWLPCAKTLAGKLEEEQIKNRAASLPIVVCHGKADDVVPFKFGEKSSQALLSNGFKKVTFKPYSALGHHTIPQELDELCAWLTSTLSLEG

SEQ ID NO: 15 - DNA, 18 bp, Arabidopsis thaliana
cccatggcttcttcactg

SEQ ID NO: 16 - DNA, 24 bp, Arabidopsis thaliana
actagtccctttatgtatccactg

SEQ ID NO: 17 - protein, 384 aa, Arabidopsis thaliana
MASSLKKLISSFLLVLYSTTIIVASSESRCRRFKSIISFGDSIADTGNYLHLSDVNHLPQSAFLPYGESFFHPPSGRASNGRLIIDFIAEFLGLPYVPPYFGSQNVSFEQGINFAVYGATALDRAFLLGKGIESDFTNVSLSVQLDTFKQILPNLCASSTRDCKEMLGDSLILMGEIGGNDYNYPFFEGKSINEIKELVPLIVKAISSAIVDLIDLGGKTFLVPGGFPTGCSAAYLTLFQTVAEKDQDPLTGCYPLLNEFGEHHNEQLKTELKRLQKFYPHVNIIYADYHNSLYRFYQEPAKYGFKNKPLAACCGVGGKYNFTIGKECGYEGVNYCQNPSEYVNWDGYHLTEAAYQKMTEGILNGPYATPAFDWSCLGSGTVDT

SEQ ID NO: 18 - protein, 404 aa, Artificial Sequence
  Bovine pregastric esterase cloned by IPG with native leader replaced by the citrus P12 leader and two amino acids added to form an ER retention signal sequence
MGVGTKVLVITTMAICLISSAAYAFLGKIAKNPEASMNVSQMISYWGYPSEMHKVITADGYILQVYRIPHGKNNANHLGQRPVVFLQHGLLGSATNWISNLPKNSLGFLLADAGYDVWLGNSRGNTWAQEHLYYSPDSPEFWAFSFDEMAEYDLPSTIDFILRRTGQKKLHYVGHSQGTTIGFIAFSTNPTLAEKIKVFYALAPVATVKYTKSLFNKLALIPHFLFKIIFGDKMFYPHTFLEQFLGVEMCSRETLDVLCKNALFAITGVDNKNFNMSRLDVYIAHNPAGTSVQNTLHWRQAVKSGKFQAFDWGAPYQNLMHYHQPTPPIYNLTAMNVPIAVWSADNDLLADPQDVDLLLSKLSNLIYHKEIPNYNHLDFIWAMDAPQEVYNEIVSLMAEDKKEL

SEQ ID NO: 19 - DNA, 1215 bp, Artificial Sequence
  Bovine pregastric esterase with P12 leader coding sequence and ER retention signal sequence added
atgggtgtaggcacaaaagttctggtgatcacaactatggccatatgcctaattagctcagctgcatacgccttccttggaaaaattgctaagaaccctgaagccagtatgaatgttagtcagatgatttcctactggggctacccaagtgagatgcataaagttataactgcggatggttatatccttcaggtctatcggattcctcatggaaagaataatgctaatcatttaggtcagagacctgttgtgtttctgcagcatggtcttcttggatcagccacaaactggatttccaacctgcccaagaacagcctgggcttcctcctggcagatgctggttatgacgtgtggctggggaacagcagaggaaacacctgggcccaggaacatttatactattcaccagactccccggaattctgggctttcagctttgatgaaatggctgaatatgaccttccatctacaattgatttcatcttaaggagaacaggacagaagaagctacactatgttggccattcccaaggcaccaccattggttttatcgccttttctaccaatcccacattggctgaaaaaatcaaagtcttctatgcattagccccagttgccacagtgaagtacaccaagagcctgtttaacaaacttgcacttattcctcacttcctcttcaagattatatttggtgacaaaatgttctacccacacacttttttggaacaatttcttggtgttgaaatgtgctcccgtgagacactggatgtcctttgtaagaatgccttgtttgccattactggagttgacaataaaaacttcaacatgagtcgcttagatgtgtatatagcacataatccagcaggaacttctgttcaaaacaccctccactggagacaggctgttaagtctgggaaattccaagcttttgactggggagccccatatcagaacctaatgcattatcatcagcccacacctcccatctacaatttaacagccatgaatgtcccaattgcagtatggagtgctgacaatgacctgttggctgaccctcaggatgttgaccttctgctttcaaaactctctaatctcatttaccacaaggaaattccaaattacaatcacttggactttatctgggcaatggatgcacctcaagaagtttacaatgaaattgtttctttgatggccgaagacaaaaaggagctctag

SEQ ID NO: 20 - DNA, 23 bp, Artificial Sequence
  DNA primer used to amplify and reclone bovine PGE gene and remove ER retention signal
cgaacggctgttaagtctgggaa

SEQ ID NO: 21 - DNA, 32 bp, Artificial Sequence
  DNA primer used to amplify and reclone bovine PGE gene and remove ER retention signal
ccaactagtattactttttgtcttcggccatc

SEQ ID NO: 22 - protein, 402 aa, Artificial Sequence
  Bovine pregastric esterase with native leader replaced by the citrus P12 leader and without ER retention signal
MGVGTKVLVITTMAICLISSAAYAFLGKIAKNPEASMNVSQMISYWGYPSEMHKVITADGYILQVYRIPHGKNNANHLGQRPVVFLQHGLLGSATNWISNLPKNSLGFLLADAGYDVWLGNSRGNTWAQEHLYYSPDSPEFWAFSFDEMAEYDLPSTIDFILRRTGQKKLHYVGHSQGTTIGFIAFSTNPTLAEKIKVFYALAPVATVKYTKSLFNKLALIPHFLFKIIFGDKMFYPHTFLEQFLGVEMCSRETLDVLCKNALFAITGVDNKNFNMSRLDVYIAHNPAGTSVQNTLHWRQAVKSGKFQAFDWGAPYQNLMHYHQPTPPIYNLTAMNVPIAVWSADNDLLADPQDVDLLLSKLSNLIYHKEIPNYNHLDFIWAMDAPQEVYNEIVSLMAEDKK

SEQ ID NO: 23 - protein, 599 aa, Artificial Sequence
  Nematode triglyceride lipase-cholesterol esterase cloned by IPG, translation product, with native leader replaced by the citrus P12 leader
MGVGTKVLVITTMAICLISSAAYAENVTTPNSEEDTDMTATPSTITPLLSTAQNPSLPKTSKLPLALKTPVPTFPFSSLATSDWLSSMPTIQTLLPPPLPLTTLEVPENENFGFTSLAPLWTLPTQPPAWSPMLDSPIKPIQNSMFPTFPTMPTLPTLPTLAPFTFPTLPPPTTMKPLNITIDPEALMDVPEIITHWGYPVETHKVVTVDGYILTLHRIPHSKNETSKSASKTPKPVVFLQHGLLCTSSIWLLNLPRQSAGYIFADQGYDVWLGNMRGNTYSKEHTRMTSADRRFWKFSWEEMARYDLPAMINYALKTTKRQNLYYVGHSQGALTMFAKMSEDPEMSKKIRKFFAMAPVARMSHVKGLFQNLGQIYEQYNLVYQVFGDGEFLTNNIFTKLLTDIFCDQAVNNPLCENFIFAVSGPNSNQFNNSRIGIYLAHNPAGTSSRNILHFAQMVKKKRMSRFDHGKDLNLKIYGAPSPPEYDIRKINSSIYLFYSDFDWLANPKDVEGFLIPMLPSKTLKKATKLRDFNHNDFLWGMRARKEIYDKIINTIKLDQRRVKLQNSMERFFERQSRNPTSGLDEETMMRLRNETMNLD

SEQ ID NO: 24 - DNA, 32 bp, Artificial Sequence
  DNA primer for use in amplifying and modifying the bacterial tributyrin esterase gene encoding the native bacterial esterase so as to contain part of the P12 leader
cagctgcatacgccatggcagtaatcaatatc

SEQ ID NO: 25 - DNA, 24 bp, Artificial Sequence
  DNA primer for use in amplifying and modifying the bacterial tributyrin esterase gene encoding the native bacterial esterase so as to contain part of the P12 leader
tattaactcaatcgttcttcttgc

SEQ ID NO: 26 - DNA, 33 bp, Artificial Sequence
  DNA primer for use in amplifying and modifying the plant carboxylesterase gene encoding the native plant carboxylesterase so as to contain part of the P12 leader
cagctgcatacgccatgagtatctccggtgctg

SEQ ID NO: 27 - DNA, 24 bp, Artificial Sequence
  DNA primer for use in amplifying and modifying the plant carboxylesterase gene encoding the native plant carboxylesterase so as to contain part of the P12 leader
actagttcaaccttcgaggctgag

SEQ ID NO: 28 - DNA, 29 bp, Artificial Sequence
  DNA primer for use in amplifying and modifying the plant carboxylesterase gene encoding the native plant carboxylesterase so as to contain part of the P12 leader
cagctgcatacgccatggcttcttcactg

SEQ ID NO: 29 - DNA, 24 bp, Artificial Sequence
  DNA primer for use in amplifying and modifying the plant carboxylesterase gene encoding the native plant carboxylesterase so as to contain part of the P12 leader
actagtccctttatgtatccactg

SEQ ID NO: 30 - DNA, 20 bp, Caenorhabditis elegans
ttgcgccactatggacattg

SEQ ID NO: 31 - DNA, 20 bp, Caenorhabditis elegans
gtgagtgcaccttgtgaatg

SEQ ID NO: 32 - DNA, 22 bp, Dictyostelium discoideum
ttactactgccgtaaacattcc

SEQ ID NO: 33 - DNA, 23 bp, Dictyostelium discoideum
ttaaacatatccaccttggttac

SEQ ID NO: 34 - DNA, 27 bp, Artificial Sequence
  DNA primer for use in amplifying and modifying the amoeba acyloxyacyl hydrolase gene so as to contain part of the P12 leader
cagctgcatacgccgtaaacattccag

SEQ ID NO: 35 - DNA, 29 bp, Artificial Sequence
  DNA primer for use in amplifying and modifying the amoeba acyloxyacyl hydrolase gene so as to contain part of the P12 leader
actagtttaaacatatccaccttggttac

(19) United States
(12) Patent Application Publication
(10) Pub. No.: US 2008/0235824 A1
(43) Pub. Date: Sep. 25, 2008

(54) Use of Esterase Genes as Selectable Markers for Transforming Plant Cells

(76) Inventors: Dean W. GABRIEL, Gainesville, FL (US); Joseph D. REDDY, Alachua, FL (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/957,972

(22) Filed: Dec. 17, 2007

Related U.S. Application Data
(60) Provisional application No. 60/870,400, filed on Dec. 17, 2006.

(51) Int. Cl. C12N 15/55; A01H 5/00; C12N 15/82

(52) U.S. Cl. 800/281; 800/288; 800/278; 536/23.2; 536/24.1; 800/320.1; 800/312; 800/320; 800/320.3; 800/317.3; 800/317.4; 800/311; 800/320.2; 435/320.1

(57) ABSTRACT

Expression of esterase genes in plant cells results in the production of enzymatically active esterases that effectively resists the otherwise growth inhibitory and/or lethal effects of nonionic, fatty acid ester detergents such as Tween 20 or Span 20. Specifically, expression of a variety of esterases, including pregastric esterase, carboxyesterase, lipase and acyloxyacyl hydrolase from a wide variety of sources in plant cells is disclosed as an excellent method to protect the cells from the effects of these detergents, allowing the exposed plant cells to regenerate into whole plants in the presence of nonionic, fatty acid ester detergents, thereby providing a practical and safe method for plant transformation.

### CROSS-REFERENCE TO RELATED APPLICATIONS

This application claims priority from U.S. Provisional Patent Application No. 60/870,400, filed Dec. 17, 2006, the entire disclosure of which is hereby expressly incorporated herein by reference in its entirety for all purposes. The entire disclosure includes the specification, claims, figures and sequence listings.

### STATEMENT OF RIGHTS TO INVENTIONS MADE UNDER FEDERALLY SPONSORED RESEARCH

This invention was made without government support.

### FIELD OF THE INVENTION

This invention is in the fields of genetic engineering and plant biology.

### BACKGROUND OF THE INVENTION

All publications and patent applications herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

Permanent genetic modification of plants requires the introduction of new genetic material into the genome of a plant cell, a process called transformation. Uniform, non-chimeric, permanent genetic modification of plants requires the introduction of new genetic material into the nuclear genome of a plant cell followed by the regeneration of an entire plant from that one cell. Uniform, non-chimeric, permanent genetic modification of plants can also arise from the introduction of new genetic material into the mitochondria or chloroplasts, but since there are multiple copies of these organelles per cell, considerable additional care, involving intense selection pressure, must be used to ensure that all such organelles are direct descendants of the originally altered organelle. Most plant transformations are designed to target the nuclear genome, and require integration of the new genetic material into a chromosome, where it becomes a new, permanent, gene locus. The genetic alteration will be stably inherited by progeny of the transformed plant. Progeny can be obtained either asexually, by taking multiple cuttings of the transformed plant, or sexually, through seed. The preferred method for plant propagation depends on the species; for example, florist's geraniums are nearly always propagated asexually, while tomatoes are nearly always propagated by seed.

To accomplish this, methods must be developed to introduce DNA past several physical barriers: the plant cell wall, the cell membrane and the nuclear envelope. In addition, some method must be devised to select or screen for, transformed cells carrying the DNA of interest. Further, in order to achieve non-chimeric, whole plant transformation, plants must be entirely regenerated from a single transformed cell carrying the DNA of interest; it is not sufficient for the regenerated plant to merely contain some transformed cells among nontransformed cells. In the case of transformation into the chloroplast or mitochondrion, plants must be entirely regenerated from a single cell carrying the DNA of interest on each and every chloroplast or mitochondrion; it is not sufficient for the regenerated plant to merely contain some transformed chloroplasts or mitochondria among the plant's cells. Methods must therefore be devised to select or screen for plants that are regenerated solely from transformed cells carrying the DNA of interest, or from transformed cells with all chloroplasts or all mitochondria carrying the DNA of interest.

### SUMMARY OF THE INVENTION

Fatty acid ester detergents, including Tween 20 and Span 20, have both been found according to the present invention to strongly inhibit regeneration of plant cells and tissues into whole plants and cause cell death of both dicot and monocot cells. Inhibition of the formation of both shoots and roots was observed. This effect is relieved by providing the dicot or monocot cell with a DNA construct that expresses an esterase or lipase from any source, whether bacterial, plant or animal, thus providing a general method for efficient transformation of plant cells and regeneration of whole, nonchimeric plants that avoids the use of an antibiotic or herbicide resistance gene, and the avoids possibility of the flow of such genes into weeds. Since the Tween and Span families of detergents are readily degraded by a wide variety of esterases and lipases, a wide variety of different esterase and lipase genes from a variety of sources, including animal, plant and microbial, were found to be useful as selectable markers according to the present invention.

Thus, in one embodiment the present invention relates to an environmentally safe, effective and improved system for selection of plant cells containing nucleic acids of interest, and for regeneration of whole plants containing only cells that carry the nucleic acids of interest. In another embodiment, the invention relates to methods of plant transformation that include a selection agent, comprising a fatty acid ester detergent or wetting agent, together with an esterase gene that provides resistance to that selection agent.

In another embodiment, novel compositions and methods for selecting plant cells and regenerating plants containing only selected plant cells are provided by the present invention which provides the following: 1) a selection agent, comprising a detergent, surfactant or wetting agent with an ester bond and 2) nucleic acids encoding esterase genes operably fused with a plant promoter and terminator and providing resistance to the selection agent. In another embodiment, the present invention provides: 1) cloned esterase cDNAs from animal, plant, nematode and microbial sources; 2) operable gene fusions of the esterases to plant promoters in gene expression cassettes; 3) functional expression of enzymatically active esterases in multiple different plants and plant parts, and 4) survival and growth on plant regeneration media only of those plants carrying the esterase genes. Thus, according to the present invention, it has been discovered that esterases may be functionally expressed in plant cells to allow: 1) selection of transformed plant cells by rescuing transformed cells from the effects of fatty acid ester surfactants and 2) regeneration of said transformed plant cells into whole plants in a highly efficient manner.

In one embodiment, this invention therefore provides a safe, efficient, and generally applicable new method for the selection of transformed plants. It is one object of the present invention to, for example, utilize pregastric esterase genes and lipase genes to produce lipases that are expressed in plant cells and that provide resistance against high concentrations of these surfactants.

In one embodiment, for example, pregastric esterases are used that are nature-identical with those enzymes with a long history of commercial use in food manufacturing and which are generally recognized as safe (GRAS). For example, bovine pregastric esterase has long been used for cheese-making.

In other embodiments, an esterase gene derived, for example, from a nematode (GenBank NP_{—}504755), a bacterium (GenBank AF157601), an amoeba (GenBank AC 17075) and/or a plant cell (GenBank NP_{—}174188) are used to create selectable marker genes in gene expression cassettes.

In other embodiments, a gene expression cassette containing an esterase gene or gene fragment that functions to express active esterase in plants also has a plant secretion signal sequence that functions in plants operably fused to the amino terminus of the esterase gene or gene fragment.

In some embodiments, the present invention further provides nucleic acid molecules, operably linked to one or more expression control elements, including vectors comprising the isolated nucleic acid molecules. In one embodiment, the present invention further includes host cells transformed to contain the nucleic acid molecules of the invention and methods for producing a peptide, polypeptide or protein comprising the step of culturing a host cell transformed with a nucleic acid molecule of the invention under conditions in which the protein is expressed.

In other embodiments, this invention provides vectors comprising the nucleic acid constructs of the present invention, as well as host cells, recombinant cells and transgenic tissues and organisms comprising the vectors of the present invention. In some embodiments, this invention provides such cells and transgenic tissues and organisms that are hemizygotic, heterozygotic or homozygotic for the nucleic acid constructs, wherein if the organism is a plant it can be haploid, monoploid, diploid or polyploid. It is an object of the present invention in some embodiments to provide such cells, transgenic tissues and transgenic plants wherein they express a single copy or multiple copies of one or more lipase proteins, or lipase-like ortholog protein products of the present invention. In some embodiments it is an object of the present invention to provide a sufficiently strong selection pressure for chloroplast or mitochondrial, as well as nuclear, transformation.

According to some embodiments of the present invention it is possible to impart into virtually all plants resistance, or increased resistance, to fatty acid ester detergents, surfactants and wetting agents, including, but not limited to the Tween and Span families of nonionic detergents. There is a particular demand for the efficient generation of transgenic crop plants, both agronomic as well as horticultural, both for food crop use as well as ornamental. There is also a particular demand for the elimination of herbicide and antibiotic resistance in said transgenic plants.

In some embodiments, the present invention therefore also relates to a method for preparing transformed plant cells and plants, including seeds and all parts of plants. Multiple methods are used by those skilled in the art for introducing esterase genes into plants or plant cells of dicots or monocots, including, but not limited to, use of Agrobacterium tumefaciens and various Ti-plasmid variations, use of electroporation, particle bombardment, fibrous silicon carbide whiskers or nonfibrous silicon carbide powder. Multiple methods are available to those skilled in the art for the regeneration of fully transgenic plants, including both dicots and monocots.

In some embodiments, the present invention further provides nucleic acid probes for the detection of expression of the esterase or esterase-like proteins of the present invention, or mutants, or homologs, or orthologs thereof, in for example, plants which either have been genetically altered to express at least one of said proteins or which may naturally express esterase or esterase-like proteins, or mutants, or homologs, or orthologs thereof.

### BRIEF DESCRIPTION OF THE FIGURES

FIG. 1 shows a diagram of the modular, binary plant transformation vector pIPG833. The bovine lipase gene insert with P12 leader (labeled “P12::BL” in the Figure) is readily replaced by other lipases by recloning into the restriction endonuclease sites indicated in FIG. 1. pIPG833 does not have a selectable marker or any other gene between the right and left T-DNA borders, except for the CAMVS2 driven P12-Bovine PGE gene driven by the CAMVS2 promoter. The vector carries an origin of replication, partition locus for stable plasmid maintenance and a resolvase from pVS1 (Heeb et al., 2000) for stable replication in A. tumefaciens and integration into the Ti helper plasmid, together with a colE1 replicon for replication in E. coli, a kanamycin resistance gene for bacterial selection, and a mobilization (“mob”) region for conjugal transfer from E. coli to A. tumefaciens.

FIG. 2 shows the phenol red lipase/esterase assay using either Commercial porcine pancreatic lipase (labeled “SL”) or plant expressed bovine pregastric esterase (labeled “768 WI”). In this Figure, the first tube on the left (labeled “Buffer”) contains phenol red buffer and Tween 20 (the dark red color appears dark in this black and white figure); the second tube to the right (“SL”) has 4 Units of Sigma porcine pancreatic lipase added (the bright yellow color appears light); the third tube to the right (labeled “731”) has a twenty microliter droplet of a crude tomato plant leaf extract from leaves inoculated 4 days earlier with GV2260 carrying “empty” (ie., no lipase gene) expression vector pIPG731 (the dark red color appears dark), and the fourth tube to the right (“768 WI”) has a twenty microliter droplet of crude tomato plant leaf extract from leaves inoculated four days earlier with GV2260 carrying cloned bovine pregastric esterase expressed in pIPG768 (the bright yellow color appears light). Tomato leaf tissue particles may be seen floating in the third and fourth tubes to the right. In these assays, only the porcine lipase positive control and plant tissue inoculated with pIPG746 (cloned bovine PGE) exhibited the color change from red (dark in this black and white figure) to bright yellow (light in this black and white figure) in the indicator tubes. The photo was taken 16 hrs. after start of the assay.

FIG. 3 shows use of 1% Tween 20 for selection of transgenic tomato plants prior to the stage of shoot or root formation using the bovine PGE gene cloned in vector pIPG768 as a selectable marker. The lower three plates are transformed, regenerating leaf pieces expressing the bovine PGE gene on pIPG768 on tomato shoot regeneration medium with 1% Tween 20 added. The upper three plates are dying leaf pieces treated with “empty” vector (ie., without lipase gene) pIPG731 on the same medium.

FIG. 4 shows a 0.8% agarose gel loaded with amplified PCR products from representative nontransgenic and transgenic plants of three indicated dicots and one monocot transformed using two different esterase genes as the only selectable markers and using two different fatty acid ester detergents. The PCR primers used to amplify the P12::PGE gene from plants transformed using pIPG833 were: IPG952 (SEQ ID NO.: 1) and IPG953 (SEQ ID NO.: 2). The PCR primers used to amplify the P12::nematode lipase gene from plants transformed using pIPG875 were IPG896 (SEQ ID NO.: 30) and IPG 898 (SEQ ID NO.: 31). From left to right: M, 1 kb ladder; 1, 2 and 3, tomato var. Micro-Tom; 4, 5 and 6, tobacco var. Xanthi; 7, 8 and 9, geranium var. Avenida; 10, 11 and 12, rice var. TP-309; 13, 14 and 15, tobacco var. xanthi. Lanes 1, 4, 7, 10 and 13 were PCR products of nontransgenic control plants. Lanes 2, 3, 5, 6, 8, 9, 11 and 12 were PCR products of transgenic plants transformed using pIPG833. Lanes 14 and 15 were PCR products of transgenic tobacco transformed using pIPG875.

FIG. 5 shows use of 1% Tween 20 for confirmational screening of transgenic tomato plants carrying the bovine PGE gene cloned in vector pIPG768 after the stage of shoot and root formation. The plant on the left, labeled “731”, was transformed using kanamycin selection and the kanamycin resistance gene driven by the 35S promoter on pIPG731 (ie., “empty” vector, without a lipase gene). The plant on the right, labeled “768”, was similarly transformed using kanamycin selection and the kanamycin resistance gene driven by the 35S promoter on pIPG768 (carrying a lipase gene). Both plants were rooted, kanamycin resistant transformants. Both plants, with roots removed to ca. 1-4 mm from the base, were transferred to rooting medium containing 1% Tween 20. After one week, the roots of both plants had grown to about the extent of the pIPG731 transformed plant, and the small ink marks were placed on the plates to indicate the extent of the root growth. Two weeks later, the roots on the plant transformed with pIPG731 had ceased to elongate and its roots started to appear brown at the growing tips, while the roots on the plant transformed with pIPG768 continued growing.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. The DNA cloning techniques used in the present invention are conventional and can be performed by anyone skilled in the art, using methods taught by, for example, Sambrook et al (1989).

The present invention is based at least partly on our discovery that fatty acid ester detergents, wetting agents or surfactants can strongly inhibit the growth and regeneration of whole plants, plant parts, plant tissues and plant cells in tissue culture. Furthermore, we discovered that esterases, which may be identified by their ability to degrade such detergents, wetting agents and surfactants, may be operably expressed in plants to provide resistance to the otherwise negative growth effects of said detergents and surfactants. Furthermore, we discovered that different types of esterases from very different sources had the same protective effect in allowing selection of transgenic cells, tissues and whole plants. We also discovered that the protective effect was general in nature, and could be used on a variety of monocotyledonous and dicotyledonous plants, without limitation.

The present invention is also based, in part, on our discovery that at least some plant leader peptides, particularly those found on proteins secreted through the plant cell wall and accumulating in plant xylem tissue, potentiate the effect of esterase on survival in tissue culture and during regeneration. The present invention is also based on our discovery that these same plant leader peptides provide a means for targeting the antimicrobial effect of esterases to the plant apoplast and xylem, where they accumulate, providing a novel means of preventing chimeric growth of nontransformed cells during rooting.

The following exemplary embodiments are intended to illustrate the present invention in greater detail:

1. A fatty acid ester surfactant, detergent or wetting agent, including but not limited to a) polyoxyethylene sorbitan fatty acid esters, such as Tween 20, Span20; b) polyether phosphate esters, such as Triton QS-15; c) alkylarylpolyethelene glycol esters, such as found in “Co-Mix” (Coastal Agribusiness, Greenville, N.C.); d) alkyl aryl polyoxyethylene phosphate esters, such as found in “Comp-Ad” (Plant Health Technologies, Boise, Id.); e) alkyl phenol polyether phosphate esters, such as found in “Convert” (Precision Labs, Waukegon, Ill.); f) esters of alkylpolyethylene ethers, such as found in “E-Z Mix” (Loveland Products, Greeley, Colo.); g) alkyl aryl phosphate esters, such as found in “Embrece” (Wilbur-Ellis Co., San Francisco, Calif.); h) aliphatic phosphate esters, such as found in “Hi-Comp” (United Suppliers, Eldora, Iowa); i) phosphate esters of polyoxyethanol, such as found in “Justice” (Jay-Mar, Plover, Wis.), and j) alkyl aryl polyethoxy ethanol phosphate esters, such as found in “Mix-All Compatibility Agent” (Rosen's, Inc., Fairmont, Minn.) is used in sufficient concentration to kill plant cells and tissues in plant tissue culture medium. The surfactant, detergent or wetting agent is tested at various concentrations for the minimum inhibitory concentration needed to completely inhibit growth and/or regeneration of the plant of interest. Preferred embodiments for tobacco and tomato are 1% Tween 20 or 1% Span 20. A preferred embodiment for geranium is 0.5% Span 20. A preferred embodiment for rice is 0.5% Span 20.

2. A prokaryotic DNA or eukaryotic cDNA clone of an active esterase is obtained. A variety of methods may be used, including: 1) directly synthesizing the gene based on a known sequence; 2) synthesizing DNA primers based on a known sequence and using PCR to amplify a cDNA clone from RNA extracted from appropriate eukaryotic tissue, and 3) identifying a esterase clone from a library that is expressed in an appropriate bacteria or fungus, based on production of esterase by one of the clones on an agar indicator plate. Examples of active esterases useful for tobacco, tomato, geranium, citrus and rice include cloned pregastric esterase (PGE), nematode triglyceride lipase-cholesterol esterase, bacterial tributyrin esterase and/or plant SGNH-motif lipase. The “SGNH” motif refers to the amino acid sequence Serine, Glycine, Asparagine and Histidine.

3. An esterase clone is operably fused within a plant gene expression cassette, minimally comprising a promoter that is functional in plants, followed by the esterase clone and followed by a plant terminator in a plant expression vector that may be used for transient gene expression in plants. Several plant promoters and promoters from plant viruses that are functional in plants are widely available for use to functionally express a foreign gene in plants in transient expression assays, for example, the CaMV promoter found in the pCAMBIA series of plant expression vectors (Cambia, Can berra, Australia). Several plant terminators are also available, including the widely available NOS terminator, also found in the pCAMBIA plant expression vector series. For transfer into plant cells, the pCAMBIA plant expression vectors also contain T-DNA borders and ability to replicate in Agrobacterium tumefaciens.

4. In one embodiment, particularly in monocots, an intron may be used to increase gene expression. Introns are known to be required for abundant expression of many genes in plants, including both dicots and especially monocots, possibly by enhancing transcript stability or facilitating mRNA maturation (Callis et al., 1987; Mun, J. H. et al. 2002; Rose & Beliakoff, 2000; Rose, 2002, Simpson & Filipowicz, 1996).

5. In another embodiment, a plant secretion signal is added to the esterase coding region, replacing the native secretion signal, if any. Some plant stress-associated and/or disease-associated proteins have been found to accumulate preferentially and most abundantly in the xylem of plants, presumably requiring a specific secretion signal sequence. Only a very few proteins are found in the xylem; it is unclear how they are secreted through the plant cell wall to reach the xylem. Such proteins have secretion signal peptides that we have discovered are useful for targeting antimicrobial compounds to the plant apoplast and xylem; we call these “xylem secretion signal peptides”. For example, we found that a 24 amino acid plant signal peptide derived from one such protein, P12 (GenBank Accession # AF015782; Ceccardi et al., 1998) is useful for the purpose. The xylem secretion signal peptide sequence is amplified from an appropriate plant source by PCR and cloned upstream of the esterase sequence.

6. Plant expression of an active, correctly folded esterase that is likely to work against a selective agent may be verified in any one of several plant species using transient gene expression (Wroblewski et al. 2005). The plant expression vector carrying the esterase gene cloned in the gene expression cassette is transformed into A. tumefaciens, and the resulting transformed cells are inoculated into plants by flooding a sizeable area of leaf tissue with diluted cell cultures. An empty vector control, consisting of the plant expression vector but without the esterase gene cloned in the expression cassette, is also inoculated, preferably on the same leaf. After 3-4 days, the plant tissue that has been inoculated is ground in 200 mM NaCl, clarified by centrifugation, and assayed against the selective agent chosen in step 1 as being inhibitory to growth and/or regeneration of plant cells of interest using a sensitive esterase assay such as the one specified in Gupta et al., (2003). Esterase levels in the tissues inoculated with the esterase clone are compared with esterase levels in the tissues inoculated with the empty vector control.

7. Enzymatically active DNA constructs are then tested by permanent transformation of plant cells, both monocots and dicots, followed by regeneration and propagation of transformed plants of the desired dicot and monocot species.

It is one object of the invention to provide a general selection method for both monocot and dicot plants, independent of which particular direct or indirect transformation method is used.

### DEFINITIONS

As used herein, the term “surfactant” is a shortened version of the phrase ““surface acting agent”, and refers to any amphipathic organic molecule, meaning one that contains both hydrophobic and hydrophilic groups, that is a chemical wetting agent. The term surfactant was coined by Antara Products in 1950.

As used herein, the term “wetting agent” refers to any chemical agent that lowers the surface tension of a liquid, allowing easier spreading, and lowering the interfacial tension between two liquids.

As used herein, the term “detergent” is a general term that refers to any compound or mixture of compounds useful in cleaning, including soaps, wetting agents and surfactants.

As used herein, the term “esterase” refers inclusively to any enzyme categorized as EC 3.1.1.x, including, without limitation, carboxylesterases (EC 3.1.1.1), lipases (EC 3.1.1.3), phospholipases (EC 3.1.1.4, EC 3.1.1.32), lysophospholipases (EC 3.1.1.5), phosphatidylinositol deacylase (EC 3.1.1.52) and acyloxyacyl hydrolases (EC 3.1.1.77).

As used herein, the term “esterase-like” protein or peptide refers to any amino acid sequence that is predicted by sequence analysis of a protein or peptide coding region to encode an esterase.

As used herein, the term “carboxylic-ester hydrolase” (EC 3.1.1.1), refers to a “carboxylesterase” and catalyzes the reaction of a carboxylic ester+H₂O to an alcohol plus a carboxylate, with a preference for water soluble substrates Other common names for carboxylic-ester hydrolases are: ali-esterase; B-esterase; monobutyrase; cocaine esterase; procaine esterase; methylbutyrase; vitamin A esterase; butyryl esterase; carboxyesterase; carboxylate esterase; carboxylic esterase; methylbutyrate esterase; triacetin esterase; carboxyl ester hydrolase; butyrate esterase; methylbutyrase; carboxylesterase; propionyl esterase; nonspecific carboxylesterase; esterase D; esterase B; esterase A; serine esterase; carboxylic acid esterase; and cocaine esterase.

As used herein, the term “lipase” refers to any triacylglycerol acylhydrolase (EC 3.1.1.3), commonly called “triacylglycerol lipase” and catalyzes the reaction of triacylglycerol plus H₂O to diacylglycerol plus a carboxylate, and prefers water insoluble substrates. Other common names for lipases are: tributyrase; butyrinase; glycerol ester hydrolase; tributyrinase; Tween hydrolase; steapsin; triacetinase; tributyrin esterase; Tweenase; amino N-AP; Takedo 1969-4-9; Meito MY 30; Tween esterase; GA 56; capalase L; triglyceride hydrolase; triolein hydrolase; tween-hydrolyzing esterase; amano CE; cacordase; triglyceridase; triacylglycerol ester hydrolase; amano P; amano AP; PPL; glycerol-ester hydrolase; GEH; meito Sangyo OF lipase; hepatic lipase; lipazin; post-heparin plasma protamine-resistant lipase; salt-resistant post-heparin lipase; heparin releasable hepatic lipase; amano CES; amano B; tributyrase; triglyceride lipase; liver lipase; and hepatic monoacylglycerol acyltransferase.

As used herein, the term “phospholipase” refers to any phosphatidylcholine 2-acylhydrolase (EC 3.1.1.4) and catalyzes the reaction of phosphatidylcholine+H₂O to yield 1-acylglycerophosphocholine+a carboxylate. Other common names for phospholipases include: lecithinase A; phosphatidase; phosphatidolipase, and phospholipase A. The term “phospholipase” also refers to any phosphatidylcholine 1-acylhydrolase (EC 3.1.1.32) and catalyzes the reaction of phosphatidylcholine+H₂O=2-acylglycerophosphocholine+a carboxylate.

AS used herein, the term “lysophospholipase” refers to any 2-lysophosphatidylcholine acylhydrolase (EC 3.1.1.5) and catalyzes the reaction of 2-lysophosphatidylcholine+H₂O to yield glycerophosphocholine+a carboxylate. Other common names for lysophospholipases include: lecithinase B; lysolecithinase; phospholipase B; lysophosphatidase; lecitholipase; phosphatidase B; lysophosphatidyicholine hydrolase; lysophospholipase A1; lysophopholipase L2; lysophospholipase transacylase; neuropathy target esterase; NTE; NTE-LysoPLA and NTE-lysophospholipase.

As used herein, the term “phosphatidylinositol deacylase” refers to any 1-phosphatidyl-D-myo-inositol 2-acylhydrolase (EC 3.1.1.52) and catalyzes the reaction of 1-phosphatidyl-D-myo-inositol+H₂O to yield 1-acylglycerophosphoinositol+a carboxylate. Other common names include: phosphatidylinositol phospholipase A₂ and phospholipase A₂

As used herein, the term “acyloxyacyl hydrolase” refers to any acyloxyacyl hydrolase (EC 3.1.1.77) and catalyzes the reaction of any 3-(acyloxy)acyl group of any bacterial lipopolysaccharide to yield a 3-hydroxyacyl group of said bacterial lipopolysaccharide plus a fatty acid.

As used herein, the term “lipase-like” protein or peptide refers to any amino acid sequence that is predicted by sequence analysis of a protein or peptide coding region to encode a lipase.

As used herein, the term “allele” refers to any of several alternative forms of a gene.

As used herein, the term “amino acid” refers to the aminocarboxylic acids that are components of proteins and peptides. The amino acid abbreviations are as follows: A (Ala); C (Cys); D (Asp); E (Glu); F (Phe); G (Gly); H (His); I (iso); K (Lys); L (Leu); M (Met); N (Asn); P (Pro); Q (Gln); R (Arg); S (Ser); T (Thr); V (Val); W (Trp), and Y (Tyr).

As used herein, the term “crop plant” refers to any plant grown for any commercial purpose, including, but not limited to the following purposes: seed production, hay production, ornamental use, fruit production, berry production, vegetable production, oil production, protein production, forage production, animal grazing, golf courses, lawns, flower production, landscaping, erosion control, green manure, improving soil tilth/health, producing pharmaceutical products/drugs, producing food or food additives, smoking products, pulp production and wood production.

As used herein, the term “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the terms “dicotyledon” and “dicot” refer to a flowering plant having an embryo containing two seed halves or cotyledons. Examples include tobacco; tomato; the legumes, including peas, alfalfa, clover and soybeans; oaks; maples; roses; mints; squashes; daisies; walnuts; cacti; violets and buttercups.

The term “esterase” as used in the present invention includes EC 3.1.1.X, including, without limitation, carboxylesterases (EC 3.1.1.1), lipases (EC 3.1.1.3), phospholipases (EC 3.1.1.4, EC 3.1.1.32), lysophospholipases (EC 3.1.1.5), phosphatidylinositol deacylase (EC 3.1.1.52) and acyloxyacyl hydrolases (EC 3.1.1.77). Esterases, also referred to as lipases, are enzymes that cleave triglycerides (fats, lipids, triacylglycerols or carboxylic acid esters) into carboxylic acids (fatty acids) and mono- and di-glycerides. Esterases are classified into different groups including carboxylesterases (EC 3.1.1.1) and lipases (EC 3.1.1.3), based on the acyl chain length of the ester substrate and/or activation at the oil/water but “the borderline between esterases and lipases has never been drawn with absolute certainty” (Desnuelle & Savar, 1963). However, more recent work has shown that activation at the oil/water interface is an unsuitable criterion for distinction (Verger, 1997), and that lipases are carboxylesterases with an ability to act on “long chain” acyl glycerols (Calvo & Fontecha, 2004; Gupta et al, 2003; Singh et al, 2006). This difficulty in classification is exacerbated by the fact that many esterases show a very wide range of substrate specificities and classification into a particular group is difficult due to overlapping specificities (Calero-Rueda et al., 2002). For example, the acyloxyacyl hydrolases (EC 3.1.1.77), hydrolyze acyl groups from 3-hydrolyxlacyl groups of lipid A of lipopolysaccharides. They also possesses a wide range of phospholipase and acyltransferase activities [e.g. EC 3.1.1.4 (phospholipase A₂), EC 3.1.1.5 (lysophospholipase), EC 3.1.1.32 (phospholipase A1) and EC 3.1.1.52 (phosphatidylinositol deacylase), hydrolysing diacylglycerol and phosphatidyl compounds, but not triacylglycerols (Munford & Hunter, 1992). The differences among these groups are more along the lines of substrate preferences, not absolute substrate specificity. These enzymes are made from plant, animal, fungal and bacterial sources, and are extremely important in biotechnology, including as additives to detergents and the manufacture of foods and nutraceuticals (Jaeger & Reetz, 1998). Pregastric esterase (PGE) or lingual lipase is a major fat-digesting enzyme in newborn and in young animals, and the purified enzyme from kids exhibits both lipase and esterase characteristics (ie., hydrolyzes both short and long chain acyl glycerols) (Calvo & Fontecha, 2004). PGE is different from pancreatic lipase in not requiring emulsifiers such as bile salts. For example, milk fat globules are resistant to the action of pancreatic lipase, but they are readily hydrolyzed by PGE. The cDNA encoding bovine PGE was isolated, cloned and completely sequenced (Timmermans et al, 1994). This PGE sequence is nearly identical to all other PGEs found in mammals. Large scale production of recombinant dog gastric lipase in tobacco plants has been achieved, demonstrating that active glycosylated enzyme was produced in plants using an unmodified animal lipase coding region when operably fused with a plant promoter; notably, the antibiotic kanamycin was used for selection and the selectable marker gene nptII (neomycin phosphotransferase) was used on the transformation vector to achieve plant transformation (Gruber et al. 2001). Gruber et al. (2001) do not anticipate nor teach the use of lipase for the purpose of plant transformation (i.e., they do not teach the use of lipase as a selectable marker for the transformation of plants). In addition, transgenic maize plants producing dog gastric lipase have been produced using the herbicide bialaphos, a tripeptide composed of two alanine residues and an analogue of glutamic acid known as phosphinothricin (PPT) and the selectable marker gene bar, that acetylates the amino residue of PPT, inactivating the herbicide (Roussel et al. 2002). Roussel et al (2002) do not anticipate nor teach the use of lipase in compositions (e.g., vectors) for use in accomplishing plant transformation (i.e., as a selectable marker).

Both bovine PGE and pancreatic lipase degrade the non-ionic detergents in the Tween series, including Tweens 20, 40, 80, and 85, which are esters of lauric, palmitic, mono- and tri-oleic acids, respectively. In fact, Tween family detergents are frequently used to assay for presence of esterases and lipases, since they are readily degraded by a wide variety of esterases and lipases (Pratt et al., 2000).

As used herein, the term “female plant” refers to a plant that produces ovules. Female plants generally produce seeds after fertilization. A plant designated as a “female plant” may contain both male and female sexual organs. Alternatively, the “female plant” may only contain female sexual organs either naturally (e.g., in dioecious species) or due to emasculation (e.g., by detasselling).

As used herein, the term “filial generation” refers to any of the generations of cells, tissues or organisms following a particular parental generation. The generation resulting from a mating of the parents is the first filial generation (designated as “F1” or “F₁”), while that resulting from crossing of F1 individuals is the second filial generation (designated as “F2” or “F₂”).

As used herein, the term “gamete” refers to a reproductive cell whose nucleus (and often cytoplasm) fuses with that of another gamete of similar origin but of opposite sex to form a zygote, which has the potential to develop into a new individual. Gametes are haploid and are differentiated into male and female.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of an individual cell, cell culture, tissue, organism (e.g., a plant), or group of organisms.

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

As used herein, the term “heterologous trait” refers to a phenotype imparted to a transformed host cell or transgenic organism by an exogenous DNA segment, heterologous polynucleotide or heterologous nucleic acid.

As used herein, the term “heterozygote” refers to a diploid or polyploid individual cell or plant having different alleles (forms of a given gene) present at least at one locus.

As used herein, the term “heterozygous” refers to the presence of different alleles (forms of a given gene) at a particular gene locus.

As used herein, the terms “homolog” or “homologue” refer to a nucleic acid or peptide sequence which has a common origin and functions similarly to a nucleic acid or peptide sequence from another species.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses effected such that a gene or a combination of genes, in heterozygous (hemizygous) or homozygous condition, imparts a desired trait to the plant.

As used herein, the term “locus” (plural: “loci”) refers to any site that has been defined genetically. A locus may be a gene, or part of a gene, or a DNA sequence that has some regulatory role, and may be occupied by different sequences.

As used herein, the term “male plant” refers to a plant that produces pollen grains. The “male plant” generally refers to the sex that produces gametes for fertilizing ova. A plant designated as a “male plant” may contain both male and female sexual organs. Alternatively, the “male plant” may only contain male sexual organs either naturally (e.g., in dioecious species) or due to emasculation (e.g., by removing the ovary).

As used herein, the term “mass selection” refers to a form of selection in which individual plants are selected and the next generation propagated from the aggregate of their seeds.

As used herein, the term “monocotyledon” or “monocot” refer to any of a subclass (Monocotyledoneae) of flowering plants having an embryo containing only one seed leaf and usually having parallel-veined leaves, flower parts in multiples of three, and no secondary growth in stems and roots. Examples include lilies; orchids; rice; corn, grasses, such as tall fescue, goat grass, and Kentucky bluegrass; grains, such as wheat, oats and barley; irises; onions and palms.

As used herein, the terms “mutant” or “mutation” refer to a gene, cell, or organism with an abnormal genetic constitution that may result in a variant phenotype.

As used herein, the terms “nucleic acid” or “polynucleotide” refer to deoxyribonucleotides or ribonucleotides and polymers thereof in either single- or double-stranded form. Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. The term “nucleic acid” also encompasses polynucleotides synthesized in a laboratory using procedures well known to those skilled in the art.

As used herein, a DNA segment is referred to as “operably linked” when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers need not be contiguous with the coding sequences whose transcription they control. Linking is accomplished by ligation at convenient restriction sites or at adapters or linkers inserted in lieu thereof.

As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein, the terms “ortholog” and “orthologue” refer to a nucleic acid or peptide sequence which functions similarly to a nucleic acid or peptide sequence from another species. For example, where one gene from one plant species has a high nucleic acid sequence similarity and codes for a protein with a similar function to another gene from another plant species, such genes would be orthologs.

As used herein when discussing plants, the term “ovule” refers to the female gametophyte, whereas the term “pollen” means the male gametophyte.

As used herein, the term “phenotype” refers to the observable characters of an individual cell, cell culture, organism (e.g., a plant), or group of organisms which results from the interaction between that individual's genetic makeup (i.e., genotype) and the environment.

The term “plants” as used herein denotes complete (i.e., whole) plants and also parts of plants, including seeds, tubers, cuttings, stems, roots, shoots, leaves, petioles, branches, shoots, ears, pods, stipules, etc. The present invention has utility for any plant in the plant kingdom, whether monocotyledonous (i.e., monocot) or dicotyledonous (i.e., dicot), including but not limited to soybean, corn, alfalfa, clover, canola, sunflower, lettuce, tomato, rose, orchid, sorghum, Kentucky blue grass, rice, sudangrass, poplar tree, apple tree, eucalyptus, geranium, marigold, ginko tree, hemlock, Arabidopsis, sugarcane, egg plant, opo, squash, watermelon, rosemary, thyme, spearmint, etc.

As used herein, the term “plant line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses effected such that a gene or a combination of genes, in heterozygous (hemizygous) or homozygous condition, imparts a desired trait to the plant.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “promoter” refers to a region of DNA involved in binding RNA polymerase to initiate transcription.

As used herein, the terms “protein,” “peptide” or polypeptide” refer to amino acid residues and polymers thereof. Unless specifically limited, the terms encompass amino acids containing known analogues of natural amino acid residues that have similar binding properties as the reference amino acid and are metabolized in a manner similar to naturally occurring amino acid residues. Unless otherwise indicated, a particular amino acid sequence also implicitly encompasses conservatively modified variants thereof (e.g. conservative substitutions) as well as the sequence explicitly indicated. The term “polypeptide” also encompasses polypeptides synthesized in a laboratory using procedures well known to those skilled in the art.

As used herein, the term “recombinant” refers to a cell, tissue or organism that has undergone transformation with recombinant DNA. The original recombinant is designated as “R0” or “R₀.” Selfing the R0 produces a first transformed generation designated as “R1” or “R₁.”

As used herein, the term “self pollinated” or “self-pollination” means the pollen of one flower on one plant is applied (artificially or naturally) to the ovule (stigma) of the same or a different flower on the same plant.

As used herein, the term “signal sequence” refers to an amino acid sequence (the signal peptide) attached to the polypeptide which binds the polypeptide to the endoplasmic reticulum (“ER”) and is essential for protein secretion.

As used herein, the term “transcript” refers to a product of a transcription process.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “transformant” refers to a cell, tissue or organism that has undergone transformation. The original transformant is designated as “T0” or “T₀.” Selfing the T0 produces a first transformed generation designated as “T1” or “T₁.”

As used herein, the term “transgene” refers to a nucleic acid that is inserted into an organism, host cell or vector in a manner that ensures its function.

As used herein, the term “transgenic” refers to cells, cell cultures, organisms (e.g., plants), and progeny which have received a foreign or modified gene by one of the various methods of transformation, wherein the foreign or modified gene is from the same or different species than the species of the organism receiving the foreign or modified gene.

As used herein, the term “transposition event” refers to the movement of a transposon from a donor site to a target site.

As used herein, the term “variety” refers to a subdivision of a species, consisting of a group of individuals within the species that are distinct in form or function from other similar arrays of individuals.

As used herein, the terms “untranslated region” or “UTR” refer to any part of a mRNA molecule not coding for a protein (e.g., in eukaryotes the poly(A) tail).

As used herein, the term “vector” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

### Plant Transformation

Methods used for selection or screening of transformed cells, tissues and whole plants: 1) must be safe for the technicians or practitioners of transformation method, 2) must not introduce mutations in the donor or transforming DNA nor the recipient DNA, and 3) must not create environmental concerns, either with the process or with the resulting whole plant. By far the most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. No. 5,693,512, U.S. Pat. No. 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. See, for example, U.S. Pat. No. 7,250,554, which utilizes P-DNA instead of T-DNA in the transformation vector.

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. No. 5,204,253, U.S. Pat. No. 5,015,580). A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. However, the efficiencies of each of these indirect or direct methods in introducing foreign DNA into plant cells are invariably extremely low, making it necessary to use some method for selection of only those cells that have been transformed, and further, allowing growth and regeneration into plants of only those cells that have been transformed.

For efficient plant transformation, a selection method must be employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. No. 5,767,378; U.S. Pat. No. 5,994,629). More typically, however, negative selection is used because it is more efficient, utilizing selective agents such as herbicides or antibiotics that either kill or inhibit the growth of nontransformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. No. 5,034,322, U.S. Pat. No. 6,174,724 and U.S. Pat. No. 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet. 79: 625-631 (1990), U.S. Pat. No. 4,795,855, U.S. Pat. No. 5,378,824 and U.S. Pat. No. 6,107,549). In addition, the dhfr gene, which confers resistance to the anticancer agent methotrexate, has been used for selection (Bourouis et al., EMBO J. 2(7): 1099-1104 (1983).

A major problem with these methods is that the resulting transformed plant carries either an antibiotic resistance gene, an herbicide resistance gene or an anticancer drug resistance gene, and there is a high level of concern that widespread deployment of these antibiotic, herbicide or anticancer drug resistance genes might result in movement or gene flow of these resistance genes to other plants through cross pollination, resulting in weeds that are now resistant to herbicides, or resulting in the acquisition of antibiotic resistance genes by microbes attacking or degrading the transformed plants, resulting in antibiotic resistant bacteria. This concern is so great that a variety of methods have been devised for the removal of the antibiotic or herbicide resistance gene after selection. Some of these methods, such as “terminator” technology (U.S. Pat. No. 5,723,765), have proved highly controversial, while others (for example, U.S. Pat. No. 4,959,317) are technically cumbersome. Clearly, better initial selection methods are needed that avoid the problems inherent in the widespread deployment of antibiotic resistance genes. Esterases, such as those disclosed in the present invention that can degrade detergents, wetting agents or surfactants that kill plant cells, meet this need as demonstrated by the present invention.

The present invention is based at least partly on our discovery that fatty acid ester detergents, such as the Tween and Span series of polyoxyethylene sorbitan fatty acid ester surfactants, can strongly inhibit plant cell regeneration, and even kill plant cells growing in tissue culture in a concentration dependent manner. This inhibition and killing includes both monocots and dicots. This concept may seem counter-intuitive in plant transformation, since nonionic fatty acid ester surfactants, including Tween 20, are often used, not as selection agents, but as wetting agents to increase plant transformation efficiencies by Agrobacterium (and using antibiotics or herbicides as the selection agents; for example, refer Curtis & Nam, 2001). It is not taught or suggested in the literature that Tween or Span or other surfactants can be used as selection agents in plant transformations. Neither are detergents, surfactants or wetting agents considered to be antibiotics or herbicides. Indeed, Tween 20 has been demonstrated to increase the number of surviving transformants produced when using an herbicide for selection (Curtis & Nam, 2001). A large number of chemicals, including fatty acid ester detergents, surfactants or wetting agents will kill plant tissue when applied in sufficient concentration, as taught in the present invention.

In principal, any fatty acid ester detergent, of which there are many, may be used as a selection agent in plant transformation, provided an esterase/lipase gene is found that can be expressed in plants as a selectable marker to produce an active esterase/lipase enzyme that degrades the fatty acid ester detergent. For example, besides the non-ionic Tween and Span families of polyoxyethylene sorbitan fatty acid ester detergents, there are non-ionic fatty acid esters of polyhydric alcohols such as polyethylene glycol (PEG), including but not limited to PEG mono- and di-laurates, such as Emanon (Kao Chemical Corp., Japan), PEG mono- and di-stearates, PEG mono- and di-oleates, and both cationic (eg., Claffey et al., 2000) and anionic (eg., Abd Maurad et al., 2006) fatty acid ester detergents, such as the Triton QS family.

Feng et al (U.S. Pat. No. 6,107,549) disclose a method for providing resistance against pyridine herbicides using a specific group of esterases. They do not teach or suggest that esterases can be used to provide a plant transformation method using a compound other than a pyridine herbicide, nor do they teach or suggest that esterases can be used to provide resistance against surfactants, detergents or wetting agents used as selective agents. Nowhere in the literature is it taught or suggested that surfactants, detergents or wetting agents are herbicidal or can be used as herbicidal agents. Indeed, fatty acid ester surfactants, detergents or wetting agents, including Tween 20, are widely used as additives to foliar crop sprays to enhance uniform coating of plants by fungicides in order to better protect crops from fungal attack (Kim et al. 2004; Reuvini et al. 1995). Nowhere in the literature is it taught or suggested that the enzymatic action of lipases or esterases could directly or indirectly be useful in the transformation of plant cells using detergents, surfactants or wetting agents as the selective agent. Nowhere in the literature is it taught or suggested that lipase or esterase genes could be used as selectable markers in the transformation of plant cells using detergents, surfactants or wetting agents as the selective agents.

As discussed herein, several embodiments of the present invention employ expression units (or expression vectors or systems) to express an exogenously supplied nucleic acid sequence in a plant. Methods for generating expression units/systems/vectors for use in plants are well known in the art and can readily be adapted for use in the instant invention. A skilled artisan can readily use any appropriate plant/vector/expression system in the present methods following the outline provided herein.

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make expression units for use in the present invention. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Lastly, plant promoters such as prolifera promoter, fruit-specific promoters, Ap3 promoter, heat shock promoters, seed-specific promoters, etc. can also be used.

Either a gamete-specific promoter, a constitutive promoter (such as the CaMV or Nos promoter), an organ-specific promoter (such as the E8 promoter from tomato) or an inducible promoter is typically ligated to the protein or antisense encoding region using standard techniques known in the art. The expression unit may be further optimized by employing supplemental elements such as transcription terminators and/or enhancer elements.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a preexisting vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835-846 (1984)) or the nopaline synthase signal (Depicker et al., Mol. and Appl. Genet. 1:561-573 (1982)).

The resulting expression unit is ligated into or otherwise constructed to be included in a vector that is appropriate for higher plant transformation. One or more expression units may be included in the same vector. The vector will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. The object of the present invention is to use an esterase or lipase gene as the selectable marker, allowing removal of all other marker genes, such as antibiotic or herbicide resistance markers. After transforming the plant cells, those cells having the vector will be identified by their ability to grow on a medium containing the esterase or lipase gene. Replication sequences, of bacterial or viral origin, are generally also included to allow the vector to be cloned in a bacterial or phage host, preferably a broad host range prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

The sequences of the present invention can also be fused to various other nucleic acid molecules such as Expressed Sequence Tags (ESTs), epitopes or fluorescent protein markers.

ESTs are gene fragments, typically 300 to 400 nucleotides in length, sequenced from the 3′ or 5′ end of complementary-DNA (cDNA) clones. Nearly 30,000 Arabidopsis thaliana ESTs have been produced by a French and an American consortium (Delseny et al., FEBS Lett. 405(2):129-132 (1997); Arabidopsis thaliana Database, http://genome.www.stanford.edu/Arabidopsis). For a discussion of the analysis of gene-expression patterns derived from large EST databases, see, e.g., M. R. Fannon, TIBTECH 14:294-298 (1996).

Biologically compatible fluorescent protein probes, particularly the self-assembling green fluorescent protein (GFP) from the jellyfish Aequorea Victoria, have revolutionized research in cell, molecular and developmental biology because they allow visualization of biochemical events in living cells (Murphy et al., Curr. Biol. 7(11):870-876 (1997); Grebenok et al., Plant J. 11(3):573-586 (1997); Pang et al., Plant Physiol. 112(3) (1996); Chiu et al., Curr. Biol. 6(3):325-330 (1996); Plautz et al., Gene 173(1):83-87 (1996); Sheen et al., Plant J. 8(5):777-784 (1995)).

Site-directed mutagenesis has been used to develop a more soluble version of the codon-modified GFP called soluble-modified GFP (smGFP). When introduced into Arabidopsis, greater fluorescence was observed when compared to the codon-modified GFP, implying that smGFP is ‘brighter’ because more of it is present in a soluble and functional form (Davis et al., Plant Mol. Biol. 36(4):521-528 (1998)). By fusing genes encoding GFP and beta-glucuronidase (GUS), researchers were able to create a set of bifunctional reporter constructs which are optimized for use in transient and stable expression systems in plants, including Arabidopsis (Quaedvlieg et al., Plant Mol. Biol. 37(4):715-727 (1998)).

Berger et al. (Dev. Biol. 194(2):226-234 (1998)) report the isolation of a GFP marker line for Arabidopsis hypocotyl epidermal cells. GFP-fusion proteins have been used to localize and characterize a number of Arabidopsis genes, including geranylgeranyl pyrophosphate (GGPP) (Zhu et al., Plant Mol. Biol. 35(3):331-341 (1997).

To introduce a desired gene or set of genes by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as resistance to an insect pest, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736369; Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

Agrobacterium tumefaciens is a naturally occurring bacterium that is capable of inserting its DNA (genetic information) into plants, resulting in a type of injury to the plant known as crown gall. Most species of plants can now be transformed using this method, including alfalfa. See, for example, Wang et al., Australian Journal of Plant Physiology 23(3): 265-270 (1996); Hoffman et al., Molecular Plant-Microbe Interactions 10(3): 307-315 (1997); and, Trieu et al., Plant Cell Reports 16:6-11 (1996).

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic® Gene Gun was invented in 1983-1984 at Cornell University by john Sanford, Edward Wolf, and Nelson Allen. It and its registered trademark are now owned by E. I. du Pont de Nemours and Company. Most species of plants have been transformed using this method, including alfalfa (U.S. Pat. No. 5,324,646) and clover (Voisey et al., Biocontrol Science and Technology 4(4): 475-481 (1994); Quesbenberry et al., Crop Science 36(4): 1045-1048 (1996); Khan et al., Plant Physiology 105(1): 81-88 (1994); and, Voisey et al., Plant Cell Reports 13(6): 309-314 (1994)).

Developed by ICI Seeds Inc. (Garst Seed Company) in 1993, WHISKERST™ is an alternative to other methods of inserting DNA into plant cells (e.g., the Biolistic® Gene Gun, Agrobacterium tumefaciens, the “Shotgun” Method, etc.); and it consists of needle-like crystals (“whiskers”) of silicon carbide. The fibers are placed into a container along with the plant cells, then mixed at high speed, which causes the crystals to pierce the plant cell walls with microscopic “holes” (passages). Then the new DNA (gene) is added, which causes the DNA to flow into the plant cells. The plant cells then incorporate the new gene(s); and thus they have been genetically engineered.

The essence of the WHISKERS™ technology is the small needle-like silicon carbide “whisker” (0.6 microns in diameter and 5-80 microns in length) which is used in the following manner. A container holding a “transformation cocktail” composed of DNA (e.g., agronomic gene plus a selectable marker gene), embryogenic corn tissue, and silicon carbide “whiskers” is mixed or shaken in a robust fashion on either a dental amalgam mixer or a paint shaker. The subsequent collisions between embryogenic corn cells and the sharp silicon carbide “whiskers” result in the creation of small holes in the plant cell wall through which DNA (the agronomic gene) is presumed to enter the cell. Those cells receiving and incorporating a new gene are then induced to grow and ultimately develop into fertile transgenic plants.

Silicon carbide “whisker” transformation has now produced stable transformed calli and/or plants in a variety of plants species such as Zea mays. See, for example, U.S. Pat. Nos. 5,302,523 and 5,464,765, each of which is incorporated herein by reference in their entirety; Frame et al., The Plant Journal 6: 941-948 (1994); Kaeppler et al., Plant Cell Reports 9:415-418 (1990); Kaeppler et al., Theoretical and Applied Genetics 84:560-566 (1992); Petolino et al., Plant Cell Reports 19 (8):781-786 (2000); Thompson et al., Euphytica 85:75-80 (1995); Wang et al., In Vitro Cellular and Developmental Biology 31:101-104 (1995); Song et al., Plant Cell Reporter 20:948-954 (2002); Petolino et al., Molecular Methods of Plant Analysis, in Genetic Transformation of Plants, Vol. 23, pp. 147-158, Springer-Verlag, Berlin (2003). Other examples include Lolium multiflorum, Lolium perenne, Festuca arundinacea, Agrostis stolonifera (Dalton et al., Plant Science 132:31-43 (1997)), Oryza sativa (Nagatani et al., Biotechnology Techniques 11:471-473 (1997)), and Triticum aestivum and Nicotiana tobacum (Kaeppler et al., Theoretical and Applied Genetics 84:560-566 (1992)). Even Chlamydomonas (see, for example, Dunahay, T. G., Biotechniques 15:452-460 (1993)) can be transformed with a “whiskers” approach. As it is currently practiced on higher plants, the “whisker” system is one of the least complex ways to transform some plant cells.

Genes successfully introduced into plants using recombinant DNA methodologies include, but are not limited to, those coding for the following traits: seed storage proteins, including modified 7S legume seed storage proteins (see, for example, U.S. Pat. Nos. 5,508,468, 5,559,223 and 5,576,203); herbicide tolerance or resistance (see, for example, De Greef et al., Bio/Technology 7:61 (1989); U.S. Pat. No. 4,940,835; U.S. Pat. No. 4,769,061; U.S. Pat. No. 4,975,374; Marshall et al. (1992) Theor. Appl. Genet. 83, 435; U.S. Pat. No. 5,489,520; U.S. Pat. No. 5,498,544; U.S. Pat. No. 5,554,798; Powell et al., Science 232:738-743 (1986); Kaniewski et al., Bio/Tech. 8:750-754 (1990)); Day et al., Proc. Natl. Acad. Sci. USA 88:6721-6725 (1991)); phytase (see, for example, U.S. Pat. No. 5,593,963); resistance to bacterial, fungal, nematode and insect pests, including resistance to the lepidoptera insects conferred by the Bt gene (see, for example, U.S. Pat. Nos. 5,597,945 and 5,597,946; Johnson et al., Proc. Natl. Acad. Sci. USA, 86:9871-9875 (1989); Perlak et al., Bio/Tech. 8:939-943 (1990)); lectins (U.S. Pat. No. 5,276,269); flower color (Meyer et al., Nature 330:677-678 (1987); Napoli et al., Plant Cell 2:279-289 (1990); van der Krol et al., Plant Cell 2:291-299 (1990)); Bt genes (Voisey et al., supra); neomycin phosphotransferase II (Quesbenberry et al., supra); the pea lectin gene (Diaz et al., Plant Physiology 109(4):1167-1177 (1995); Eijsden et al., Plant Molecular Biology 29(3):431-439 (1995)); the auxin-responsive promoter GH3 (Larkin et al., Transgenic Research 5(5):325-335 (1996)); seed albumin gene from sunflowers (Khan et al., Transgenic Research 5(3):179-185 (1996)); and genes encoding the enzymes phosphinothricin acetyl transferase, beta-glucuronidase (GUS) coding for resistance to the Basta® herbicide, neomycin phosphotransferase, and an alpha-amylase inhibitor (Khan et al., supra), each of which is expressly incorporated herein by reference in their entirety.

Transgenic alfalfa plants have been produced using a number of different genes isolated from both alfalfa and non-alfalfa species including, but not limited to, the following: the promoter of an early nodulin gene fused to the reporter gene gusA (Bauer et al., The Plant Journal 10(1):91-105 (1996)); the early nodulin gene (Charon et al., Proc. Natl. Acad. of Sci. USA 94(1):8901-8906 (1997); Bauer et al., Molecular Plant-Microbe Interactions 10(1):39-49 (1997)); NADH-dependent glutamate synthase (Gantt, The Plant Journal 8(3):345-358 (1995)); promoter-gusA fusions for each of three lectin genes (Bauchrowitz et al., The Plant Journal 9(1):31-43 (1996)); the luciferase enzyme of the marine soft coral Renilla reniforms fused to the CaMV promoter (Mayerhofer et al., The Plant Journal 7(6): 1031-1038 (1995)); Mn-superoxide dismutase cDNA (McKersie et al., Plant Physiology 111(4): 1177-1181 (1996)); synthetic cryIC genes encoding a Bacillus thuringiensis delta-endotoxin (Strizhov et al., Proc. Natl. Acad. Sci. USA 93(26):15012-15017 (1996)); glucanse (Dixon et al., Gene 179(1):61-71 (1996); and leaf senescence gene (U.S. Pat. No. 5,689,042).

Genetic transformation has also been reported in numerous forage and turfgrass species (Conger B. V., Genetic Transformation of Forage Grasses in Molecular and Cellular Technologies for Forage Improvement, CSSA Special Publication No. 26, Crop Science Society of America, Inc. E. C. Brummer et al. Eds. 1998, pages 49-58). These include, but are not limited to, orchardgrass (Dactylis glomerata L.), tall fescue (Festuca arundinacea Schreb.) red fescue (Festuca rubra L.), meadow fescue (Festuca pratensis Huds.) perennial ryegrass (Lolium perenne L.) creeping bentgrass (Agrostis palustris Huds.) and redtop (Agrostis alba L.).

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

Assuming normal hemizygosity, selfing will result in maximum genotypic segregation in the first selfed recombinant generation, also known as the R1 or R₁ generation. The R1 generation is produced by selfing the original recombinant line, also known as the R0 or R₀ generation. Because each insert acts as a dominant allele, in the absence of linkage and assuming only one hemizygous insert is required for tolerance expression, one insert would segregate 3:1, two inserts, 15:1, three inserts, 63:1, etc. Therefore, relatively few R1 plants need to be grown to find at least one resistance phenotype (U.S. Pat. Nos. 5,436,175 and 5,776,760).

As mentioned above, self-pollination of a hemizygous transgenic regenerated plant should produce progeny equivalent to an F2 in which approximately 25% should be homozygous transgenic plants. Self-pollination and testcrossing of the F2 progeny to non-transformed control plants can be used to identify homozygous transgenic plants and to maintain the line. If the progeny initially obtained for a regenerated plant were from cross-pollination, then identification of homozygous transgenic plants will require an additional generation of self-pollination (U.S. Pat. No. 5,545,545).

### Breeding Methods

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection. In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Hybrids. A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

It should be understood that the examples and embodiments described herein are for illustrative purposes only and that various modifications or changes in light thereof will be suggested to persons skilled in the art and are to be included within the spirit and purview of this application.

### EXAMPLES

### Example 1

### Determination of Effective Selective Agent Concentrations for Use in Transformations of Geranium

Tween 20, a nonionic, fatty acid ester detergent, was evaluated as a potential selective agent for use in geranium (Pelargonium X hortorum) transformation experiments by placing geranium petiole explants into tissue culture with the addition to Tween 20 to the regeneration (shooting) medium normally used in geranium transformations (Robichon et al., 1995). Tween 20 was added at three concentrations: 1%, 0.1% and 0.01%. Each treatment consisted of 40 sterilized petiole explants placed in four tissue culture plates carrying 10 explants each. Survival of the explants was assayed weekly over four weeks on these media. Potential selective agent Span 20, also a nonionic, fatty acid ester detergent, was similarly evaluated, except that concentrations of 1%, 0.5% and 0.25% were evaluated over a twelve week period of time. The nonionic detergent Triton X-100, which is not a fatty acid ester detergent, was included for comparative purposes. The survival results were as follows:

TABLE 1
Survival of geranium petiole explants on regeneration
medium in the presence of the indicated detergents.

| Treatment | Week 1 | Week 2 | Week 3 | Week 4 | Week 7 | Week 10 | Week 12 |
|---|---|---|---|---|---|---|---|
| 1% Tween | 10/40 | 0/40 | 0/40 | 0/40 | — | — | — |
| 20 | | | | | | | |
| 0.1% Tween | 31/40 | 10/40 | 0/40 | 0/40 | — | — | — |
| 20 | | | | | | | |
| 0.01% | 33/40 | 27/40 | 20/40 | 0/40 | — | — | — |
| Tween 20 | | | | | | | |
| 1% Span 20 | 114/114 | ND | 74/114 | ND | 11/114 | ND | 0/114 |
| 0.5% Span | 46/46 | ND | 35/46 | ND | 23/46 | ND | 5/46 |
| 20 | | | | | | | |
| 1% Triton X | 39/40 | 39/40 | 37/40 | 19/40 | ND | ND | ND |
| 0.1% Triton X | 40/40 | 40/40 | 40/40 | 9/40 | ND | ND | ND |
| 0.01% | 39/40 | 39/40 | 39/40 | 14/40 | ND | ND | ND |
| Triton X | | | | | | | |

As may be seen in Table 1, Tween 20 completely killed all geranium explants in four weeks, even at the lowest level (0.01%) tested. Span 20 was slower but still quite effective; Span 20 required twelve weeks to completely kill all geranium explants at the 1% level. Triton X was much less efficient and much more variable in killing nontransgenic geranium cells in tissue culture medium. This example demonstrates that the nonionic, fatty acid ester detergents Tween 20 and Span 20 can kill geranium cells in tissue culture, and also demonstrates a general method for determining the minimum effective concentration of a given detergent, surfactant or wetting agent in killing or inhibiting geranium tissues placed in tissue culture.

### Example 2

### Determination of an Effective Selective Agent Concentration for Use in Transformations of Tomato

Tween 20 was similarly evaluated as a potential selective agent for use in tomato (Lycoperisicon esculentum) transformations by placing tomato leaf explants placed into tissue culture; Tween 20 was added to tomato regeneration (shooting) medium normally used in tomato transformations (Riggs et al., 2001) at a 1% concentration. Each treatment consisted of 40 sterilized leaf explants placed in four tissue culture plates carrying 10 explants each. Survival of the explants was assayed weekly over four weeks on these media. The survival results were as follows:

TABLE 2
Survival of tomato leaf explants on regeneration
medium in the presence of the indicated detergents.

| Treatment | Week 1 | Week 2 | Week 3 | Week 4 |
|---|---|---|---|---|
| 1% Tween 20 | 8/40 | 8/40 | 3/40 | 0/40 |
| 1% Triton X | 40/40 | 40/40 | 10/40 | 7/40 |

As may be seen in Table 2, Tween 20 completely killed all tomato explants in four weeks. Triton X was again both less effective and unpredictable in killing. Taken together with Example 1, this example demonstrates that the nonionic, fatty acid ester detergent Tween 20 can be used to kill tomato as well as geranium cells in tissue culture, and also further demonstrates a general method for determining the minimum effective concentration of a given detergent, surfactant or wetting agent in killing or inhibiting growth of plant tissues placed in tissue culture.

### Example 3

### Determination of an Effective Selective Agent Concentration for Use in Transformations of Rice

Span 20 was evaluated as a potential selective agent for use in transformation of rice (Oryza sativa japonica var. TP-309) in a manner similar to that described in Examples 1 and 2, except that the regeneration (shooting) medium used was appropriate for regeneration of rice from callus produced from seeds (Hiei et al., 1997). Selection of rice normally requires longer periods of time than geranium or tomato. Survival results were as follows:

TABLE 3
Survival of rice grain explants on regeneration medium
in the presence of Span 20.

| Treatment | Week 4 | Week 14 | Week 20 |
|---|---|---|---|
| 1% Span 20 | 60/60 | 5/60 | 0/60 |
| 0.5% Span 20 | 60/60 | 12/60 | 2/60 |

As may be seen in Table 3, 1% Span 20 completely killed all rice explants in twenty weeks. Taken together with Examples 1 and 2, this example demonstrates that the nonionic, fatty acid ester detergent Span 20 can be used to kill cells in tissue culture of the monocot rice, as well as the dicots geranium and tomato, and also further demonstrates a general method for determining the minimum effective concentration of a given fatty acid ester detergent, surfactant or wetting agent in killing or inhibiting growth of plant tissues placed in tissue culture.

### Example 4

### Determination of an Effective Selective Agent Concentration for Use in Transformations of Tobacco

PEG 400 monolaurate, also a nonionic, fatty acid ester surfactant, was evaluated as a potential selective agent for use in transformation of tobacco (Nicotiana tabacum) in a manner similar to that described for Tween 20 and Span 20 in Examples 1, 2 and 3, except that the regeneration medium used was appropriate for regeneration of tobacco from leaf discs (Horsch et al., 1985).

### Example 5

### Determination of an Effective Selective Agent for Use in Transformations of Citrus

Triton QS-15, an anionic fatty acid ester is also evaluated as a potential selective agent for use in transformation of citrus (Citrus sinensis Osb. x Poncirus trifoliata L. Raf) in a manner similar to that described for Tween 20 and Span 20 in Examples 1, 2, 3 and 4, except that the regeneration medium used is appropriate for regeneration of citrus from etiolated seedling stem pieces (Moore et al., 1992).

### Example 6

### Use of Recombinant DNA Techniques to Obtain a Bovine Pregastric Esterase (PGE) Gene from a Natural Source

Salivary glands were removed from a freshly slaughtered calf within 15 minutes of death. RNA from the salivary glands was immediately extracted from three different glands using RNAeasy (Qiagen). cDNA was prepared from RNA extracts from two of the salivary glands using Thermoscript reverse transcriptase and PCR primers IPG451 (5′-atgcccatggaacatatgatgtggtggctacttgtaaca-3′) (SEQ ID NO.: 3) and IPG452 (5′-gcat cccggg cta gagctc ctttttgtcttcggccaqtcaa-3′) (SEQ ID NO.: 4). The primers were designed to amplify the complete Bos taurus (calf) PGE gene (GenBank Accession: L26319 [gi:600756]), and to introduce NcoI and NdeI enzymatic cloning sites upstream of the ATG translational start site, and also SmaI and SacI enzymatic cloning sites downstream of the gene. The latter sites also served to add two additional amino acids to the native gene, forming an endoplasmic reticulum (ER) retention signal sequence. Independent PCR amplifications were made. The resulting PCR products were cloned into E. coli vector pGemT. Nine independently amplified calf lipase cDNA clones were sequenced. All of these clones contained the native calf secretion signal leader sequence. Besides several clones that carried variations that were specific to the given clone and were discarded as PCR artifacts, there were three variations from the published clone that were common among the nine lipase clones. These three are either natural variations among different calves or the published sequence has three errors. The discrepancies are not likely themselves errors, because multiple clones from independent PCR reactions of two independent salivary glands were sequenced, and several of these were identical to the consensus sequence. One of these, pIPG442-108, carrying the bovine PGE gene encoding the native PGE, with native N-terminal secretion signal and additional two C-terminal amino acids forming an ER retention signal (SEQ ID No. 5), was selected for plant expression and for further manipulations. These results demonstrate that recombinant DNA could be used by one skilled in the art to readily obtain the DNA coding region for an animal esterase, provided the DNA sequence is known.

### Example 7

### Use of Recombinant DNA Techniques to Obtain a Nematode Triglyceride Lipase-Cholesterol Esterase Gene from a Natural Source

RNA was extracted from the culture-grown nematode Caenorhabditis elegans obtained from Carolina Biological Supply (Burlington, N.C.) using RNAeasy (Qiagen). cDNA was prepared from RNA extracts using Thermoscript reverse transcriptase and PCR primers IPG915 (5′-CAGCTGCATACGCcgaaaatgtcaccactcc-3′) (SEQ ID NO.: 6) and IPG894 (5′-ttacgaaatagtatctggaag-3′) (SEQ ID NO.: 7). The primers were designed to amplify the C. elegans triglyceride lipase-cholesterol esterase gene (GenBank Accession: NP_{—}504755) without a leader sequence and to introduce the PvuII enzymatic cloning site upstream of the coding region, and also the SpeI enzymatic cloning site downstream of the gene. Independent PCR amplifications were made. The resulting PCR products were cloned into E. coli vector pGemT. Four independently amplified cDNA clones were sequenced. Besides several clones that carried variations that were specific to the given clone and were discarded as PCR artifacts, the cloned gene was 98% identical to the published sequence. One of these, pIPG818, carrying the nematode triglyceride lipase-cholesterol esterase gene encoding the native nematode esterase (SEQ ID No. 8), was selected for use in plant expression vectors and for further manipulations. These results demonstrate that recombinant DNA can be used by one skilled in the art to readily obtain the DNA coding region for a nematode esterase, provided the DNA sequence is known.

### Example 8

### Use of Recombinant DNA Techniques to Obtain a Bacterial Tributyrin Esterase Gene from a Natural Source

DNA was extracted from the culture-grown, Gram positive bacterium Lactococcus lactis subsp. cremoris using standard methods. PCR primers IPG922 (5′-ccATGGCAGTAATCAATATCGAA-3′) (SEQ ID NO.: 9) and IPG923 (5′-TATTAACTCAATCGTTCTTCTTGC-3′) (SEQ ID NO.: 10) were designed and used to amplify the complete Lactococcus lactis subsp. cremoris tributyrin esterase gene (GenBank Accession: AF157601) and to introduce the NcoI enzymatic cloning site upstream of the ATG translational start site, and also the SpeI enzymatic cloning site downstream of the gene. Independent PCR amplifications were made. The resulting PCR products were cloned into E. coli vector pGemT. Four independently amplified cDNA clones were sequenced. Besides several clones that carried variations that were specific to the given clone and were discarded as PCR artifacts, the cloned gene was identical to the published sequence. One of these, pIPG849, carrying the bacterial tributyrin esterase gene encoding the native bacterial esterase (SEQ ID No. 11), was selected for use in plant expression vectors and for further manipulations. These results demonstrate that recombinant DNA could be used by one skilled in the art to readily obtain the DNA coding region for a bacterial esterase, provided the DNA sequence is known.

### Example 9

### Use of Recombinant DNA Techniques to Obtain a Plant Carboxylesterase Gene from a Natural Source

RNA was extracted from the plant Arabidopsis thaliana using RNAeasy (Qiagen). cDNA was prepared from RNA extracts using Thermoscript reverse transcriptase and PCR primers IPG970 (5′-TCatgagtatctccggtgctg-3′) (SEQ ID NO.: 12) and IPG971 (5′-ACTAGTtcaaccttcgaggctgag-3′) (SEQ ID NO.: 13). The primers were designed to amplify the complete A. thaliana carboxylesterase gene (GenBank Accession: NM_{—}203086) and to introduce the BspHI enzymatic cloning site upstream of the ATG translational start site, and also the SpeI enzymatic cloning site downstream of the gene. Independent PCR amplifications were made. The resulting PCR products were cloned into E. coli vector pGemT. Independently amplified cDNA clones were sequenced. Besides several clones that carried variations that were specific to the given clone and were discarded as PCR artifacts, the cloned gene was identical to the published sequence. One of these, pIPG876, carrying the plant carboxylesterase gene encoding the native plant carboxylesterase (SEQ ID No. 14), was selected for use in plant expression vectors and for further manipulations. These results demonstrate that recombinant DNA could be used by one skilled in the art to readily obtain the DNA coding region for a plant carboxylesterase, provided the DNA sequence is known. Taken together with Examples 6, 7 and 8, these results further demonstrate that recombinant DNA can be used by one skilled in the art to readily obtain the DNA coding region for an esterase from virtually any source—animal, plant or microbial—provided the DNA sequence is known.

### Example 10

### Use of Recombinant DNA Techniques to Obtain a Plant Lipase Gene with an SGNH Motif from a Natural Source

RNA was extracted from the plant Arabidopsis thaliana using RNAeasy (Qiagen). cDNA was prepared from RNA extracts using Thermoscript reverse transcriptase and PCR primers IPG972 (5′-cccatggcttcttcactg-3′) (SEQ ID NO.: 15) and IPG973 (5′-ACTAGTccctttatgtatccactg-3′) (SEQ ID NO.: 16). were then used to amplify the complete A. thaliana plant lipase gene with an SGNH motif (GenBank Accession: NP_{—}174188) and to introduce the NcoI enzymatic cloning site upstream of the ATG translational start site, and also the SpeI enzymatic cloning site downstream of the gene. Independent PCR amplifications were made. The resulting PCR products were cloned into E. coli vector pGemT. Independently amplified cDNA clones were sequenced. Besides several clones that carried variations that were specific to the given clone and were discarded as PCR artifacts, the cloned gene was identical to the published sequence. One of these, pIPG877, carrying the plant lipase gene encoding the native plant carboxylesterase (SEQ ID No. 17), was selected for use in plant expression vectors and for further manipulations. These results demonstrate that recombinant DNA could be used by one skilled in the art to readily obtain the DNA coding region for a plant lipase, provided the DNA sequence is known. Taken together with Examples 6, 7, 8, and 9, these results further demonstrate that recombinant DNA can be used by one skilled in the art to readily obtain the DNA coding region for virtually any esterase from virtually any source, provided the DNA sequence is known.

### Example 11

### Use of Recombinant DNA Techniques to Obtain an Amoeba Acyloxyacyl Hydrolase Gene from a Natural Source

RNA is extracted from the amoeba Dictyostelium discoideum using RNAeasy (Qiagen). cDNA is prepared from RNA extracts using Thermoscript reverse transcriptase and PCR primers IPG977 (SEQ ID NO.: 32) and IPG978 (SEQ ID NO.: 33) are then used to amplify the complete D. discoideum acyloxyacyl hydrolase gene (GenBank Accession AC117075) and to introduce the NcoI enzymatic cloning site upstream of the ATG translational start site, and also the SpeI enzymatic cloning site downstream of the gene. Independent PCR amplifications are made. The resulting PCR products are cloned into E. coli vector pGemT. Independently amplified cDNA clones were sequenced. Clones that carry variations that are specific to the given clone are discarded as PCR artifacts. The resulting clone, pIPG884, carrying the amoeba acyloxyacyl hydrolase gene is selected for use in recloning into plant expression vectors and for further DNA manipulations.

### Example 12

### Construction of a Bovine PGE with an ER Retention Signal and with a Plant Xylem Secretion Signal in an Expression Cassette in Two Plant Expression Vectors

The CaMV promoter from pBI221 (Clontech, Palo Alto, Calif.) was enzymatically recloned into the polylinker cloning site of pCAMBIA0390 (Cambia, Can berra, AU), which has a left T-DNA border, the polylinker site, a NOS transcriptional terminator and right T-DNA borders, creating pIPG700. The bovine PGE gene, including the native leader sequence and with an added ER retention signal (SEQ ID No. 5), was enzymatically recloned from pIPG442-108 into pIPG700 downstream from the CaMV promoter and upstream from the NOS terminator, creating pIPG745. A 24 amino acid plant signal peptide derived from a protein known to accumulate in the citrus xylem, P12 (GenBank Accession # AF015782; Ceccardi et al., 1998) was used to replace the native PGE leader. The xylem secretion signal peptide sequence was amplified from an appropriate plant source by PCR and cloned upstream of the mature lipase sequence, replacing the native PGE leader and resulting in a translational gene fusion between P12 and PGE (SEQ ID No. 18 and SEQ ID No. 19) on pIPG746. Clones pIPG745 and pIPG746 were used for transient expression assays in the dicots: tobacco, pepper, tomato, citrus and geranium, and in the monocot: rice (refer Example 20 below).

The P12::PGE gene with ER retention signal (SEQ ID No. 19) was enzymatically recloned from pIPG746 into pCAMBIA1305.2 (Cambia, Can berra, AU), such that the PGE gene was driven from the reverse CaMV promoter of pCAMBIA1305.2, forming pIPG774. pCAMBIA1305.2 carries the hygromycin resistance gene driven by a dual CaMV promoter for plant selection. pIPG774 was used to compare the hygromycin antibiotic resistance gene as a selectable marker against the bovine PGE gene with an ER retention signal as a selectable marker on the same plasmid in transformation experiments using geranium and rice.

The P12::PGE gene (SEQ ID No. 19) was also enzymatically recloned from pIPG746 into pCAMBIA2301 (Cambia, Can berra, AU), such that the PGE gene was driven from the reverse CaMV promoter of pCAMBIA2301, forming pIPG768. pCAMBIA2301 carries the nptII (kanamycin resistance) gene driven by a dual CaMV promoter for plant selection. pIPG768 was used both in transient expression assays and in tomato and citrus transformation experiments to compare the nptII resistance gene as a selectable marker against the bovine PGE gene with an ER retention signal as a selectable marker on the same plasmid.

### Example 13

### Construction of a Bovine PGE without an ER Retention Signal and with a Plant Xylem Secretion Signal in an Expression Cassette in a Plant Expression Vector

In order to remove the artificially added ER retention signal, a 325 bp fragment of the 3′ end of the PGE gene (SEQ ID No. 5), was PCR amplified from pIPG442-108 using IPG9295′-cgaacggctgttaagtctgggaa (SEQ ID NO.: 20) and IPG9285′-ccaactagtattactttttgtcttcggccatc (SEQ ID NO.: 21). The PCR product was digested with restriction enzymes Hind III and Spe I and ligated to pIPG442-108 digested with the same enzymes, replacing the original insert, and resulting in pIPG831. The insert in pIPG831 was verified by sequencing and digested with Apa I and Spe I to release an 846 bp internal fragment of the bovine PGE gene. This 846 bp band was ligated to pIPG768 digested with Apa I and Spe I, resulting in the elimination of the final two amino acids of Seq ID 5 and resulting in pIPG852. The bovine PGE translation product encoded on pIPG852 has a P12 signal sequence and lacks the added ER retention signal (SEQ ID No. 22). pIPG852 was used to compare the nptII (kanamycin resistance gene) as a selectable marker against the bovine PGE gene without an ER retention signal as a selectable marker on the same plasmid in transformation experiments using citrus.

### Example 14

### Construction of a Modular Plant Expression Vector with a Readily Replaceable Lipase Gene (P12::bovine PGE) for Use in Screening Different Lipase Genes in an Expression Cassette

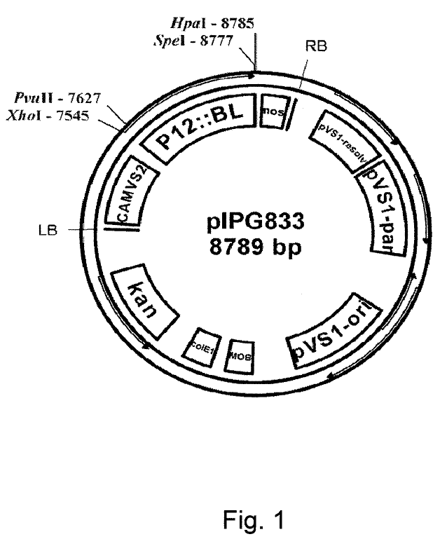

The CaMVS2 (duplicated CAMV promoter) from pCAMBIA2301 (Cambia, Can berra, AU) was enzymatically recloned into the polylinker cloning site of pCAMBIA0390, a binary vector suitable for transfer to and use in A. tumefaciens in plant transformation (Cambia, Can berra, AU), creating pIPG795. The bovine PGE gene fused to the P12 secretory leader (SEQ ID No. 22) was enzymatically recloned using BamHI and SpeI from pIPG852 into pIPG795 digested with BglII and SpeI, downstream of the CAMVS2 promoter and upstream from the NOS terminator, creating pIPG833 (FIG. 1). pIPG833 is a modular vector wherein the lipase insert is readily replaced by other lipases, simply by reckoning into the restriction endonuclease sites indicated in FIG. 1. pIPG833 does not have a selectable marker or any other gene between the right and left T-DNA borders, except for the CAMVS2 driven P12-Bovine PGE gene driven by the CAMVS2 promoter. pIPG833 was used to transform rice, geranium, tomato, tobacco and citrus with the bovine PGE gene without an ER retention signal as the sole selectable marker.

### Example 15

### Construction of a Nematode Triglyceride Lipase-Cholesterol Esterase Expression Cassette in a Plant Expression Vector

The CaMV promoter from pBI221 (Clontech, Palo Alto, Calif.) was enzymatically recloned into the polylinker cloning site of pCAMBIA0390 (Cambia, Can berra, AU), which has a left T-DNA border, the polylinker site, a NOS transcriptional terminator and right T-DNA borders, creating pIPG700. The P12 leader was cloned into pIPG700 using BamHI and SpeI, creating pIPG701. The nematode triglyceride lipase-cholesterol esterase gene in pIPG818 was excised by digesting with SpeI and PvuII. The excised fragment was ligated to pIPG701 digested with PvuII and SpeI, resulting in pIPG823, carrying a translational gene fusion between P12 and nematode triglyceride lipase-cholesterol esterase (SEQ ID No. 23). Clone pIPG823 was used for transient expression assays in tobacco and geranium to confirm expression of active, correctly folded nematode esterase.

After confirmation of nematode esterase enzymatic activity in tobacco and geranium, the nematode triglyceride lipase-cholesterol esterase with P12 leader gene was recloned, using PvuII and SpeI, from pIPG823 into the backbone of pIPG833, replacing the P12::bovine PGE gene with the P12::nematode esterase gene on the modular vector and creating pIPG875. pIPG875 does not have a selectable marker or any other gene between the right and left T-DNA borders, except for the CAMVS2 driven nematode esterase gene driven by the CAMVS2 promoter. pIPG875 was used to transform tobacco with the nematode esterase gene as the sole selectable marker.

### Example 16

### Construction of a Bacterial Tributyrin Esterase Gene Expression Cassette in a Plant Expression Vector

pIPG849 from Example 8, carrying the bacterial tributyrin esterase gene encoding the native bacterial esterase (SEQ ID No. 11), is PCR amplified using primers IPG976 (containing part of the P12 leader, SEQ ID No. 24) and IPG923 (SEQ ID No. 25), cloned into pGEM-T, and sequenced. The resulting fragment with the correct amino acid sequence is cloned into pIPG833 using PvuII and SpeI, resulting in pIPG 880. pIPG880, carrying a bacterial tributyrin esterase gene with a P12 leader sequence, is used to transform any desired plant species.

### Example 17

### Construction of a Plant Carboxylesterase Gene Expression Cassette in a Plant Expression Vector

pIPG876 from Example 9, carrying the plant carboxylesterase gene encoding the native plant carboxylesterase (SEQ ID No. 14), is PCR amplified using primers IPG974 (containing part of the P12 leader, SEQ ID No. 26) and IPG971 (SEQ ID No. 27), cloned into pGEM-T, and sequenced. The resulting fragment with the correct amino acid sequence is cloned into pIPG833 using PvuII and SpeI, resulting in pIPG881. pIPG881, carrying a plant carboxylesterase gene with a P12 leader sequence, is used to transform any desired plant species.

### Example 18

### Construction of a Plant Lipase Gene with an SGNH Motif Expression Cassette in a Plant Expression Vector

pIPG877 from Example 10, carrying the plant lipase gene encoding the native plant lipase gene with SGNH motif (SEQ ID No. 17), is PCR amplified using primers IPG975 (containing part of the P12 leader, SEQ ID No. 28) and IPG973 (SEQ ID No. 29), cloned into pGEM-T, and sequenced. The resulting fragment with the correct amino acid sequence is cloned into pIPG833 using PvuII and SpeI, resulting in pIPG882. pIPG882, carrying a plant carboxylesterase gene with a P12 leader sequence, is used to transform any desired plant species.

### Example 19

### Construction of an Amoeba Acyloxyacyl Hydrolase Gene Motif Expression Cassette in a Plant Expression Vector

pIPG884 from Example 11, carrying the amoeba acyloxyacyl hydrolase gene (GenBank AC117075) is PCR amplified using primers IPG979 (containing part of the P12 leader, SEQ ID No. 34) and IPG980 (SEQ ID No. 35), cloned into pGEM-T, and sequenced. The resulting fragment with the correct amino acid sequence is cloned into pIPG833 using PvuII and SpeI, resulting in pIPG886. pIPG886, carrying an amoeba acyloxyacyl hydrolase gene with a P12 leader sequence, is used to transform any desired plant species.

### Example 20

### Use of Transient Expression Assays in Plants to Confirm Plant Expression of Active, Correctly Folded Bovine PGE

For transient expression assays of bovine PGE, the plant transformation and expression vectors constructed in Example 12 were moved into A. tumefaciens strain GV2260 by either electroporation or bacterial conjugation as described (Kapila et al., 1997). GV2260 carrying pIPG745 or pIPG746 was used for transient expression in tobacco, pepper, tomato, citrus, geranium, and rice plants as described (Kapila et al. 1997; Duan et al., 1999; Wroblewski et al. 2005). Cultures of Agrobacterium harboring the constructs of interest were grown in minimal medium in the presence of acetosyringone to induce the Agrobacterium vir genes. The optical density of the cultures was maintained at 0.008 for pepper and tomato and at 0.25 for citrus, geranium and rice. Strain GV2260 was flooded into healthy, young, fully expanded leaves of vigorous plants into the apoplastic space through open stomata by injection using a tuberculin syringe without a needle, flooding entire leaves. After 3-4 days, two grams of leaf tissue (fresh weight), was ground in liquid nitrogen to a fine powder, and 0.5 ml of ice cold phosphate buffered saline (PBS; 137 mM NaCl, 2.7 mM KCl, 10 mM potassium phosphate, pH 7.4) was added and mixed thoroughly at 4 C. The mixture was mixed on an orbital shaker at 4 C for 30 minutes, and plant debris was pelleted at 10,000×g for 15 minutes and discarded. The supernatants from these crude plant leaf extracts were directly used in lipase/esterase assays.

Two lipase/esterase activity assays were used. The first was a rhodamine lipase assay using olive oil as a substrate as described by Jette & Ziomek (1994), with modifications. Briefly, a solution of 0.02% rhodamine (from a 2% rhodamine in ethanol stock) and 2.5% olive oil in water was mixed and sonicated thoroughly before use. The suspension was used to make agar plates, upon which 0.025 ml droplets from the crude plant extracts from leaf tissue inoculated with pIPG746 and empty vector control were placed. As a positive control, 0.025 ml droplets containing 20 U of Sigma porcine lipase was also placed, and the rhodamine agar plates incubated at 30 C overnight. Bright fluorescent spots were observed from both the porcine lipase control and from pIPG746. Lower intensity fluorescence was also observed from the empty vector controls. In order to quantify the difference, 0.2 ml of the rhodamine olive oil suspension was mixed with 0.1 ml of plant extract and allowed to incubate at 30 C for 24 hrs. Fluorescence was measured using a Tecan Spectraluor Plus spectrofluorimeter (excitation=360 nm; emission=535 nm). The results were that extracts from plant tissue inoculated with pIPG746 exhibited 17-43% higher fluorescence than the background fluorescence observed using extracts from plant tissue inoculated with the empty vector controls.

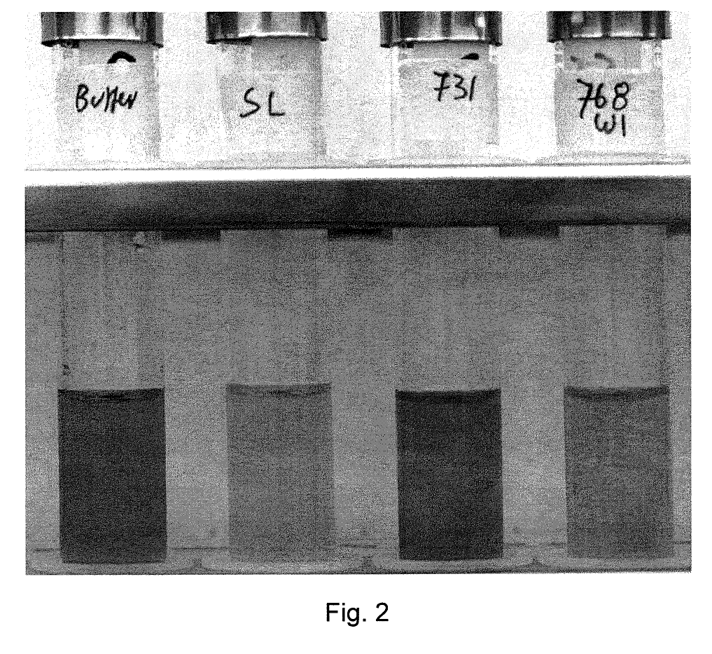

The second activity assay was a phenol red lipase/esterase assay using tributyrin, olive oil or Tween 20 as substrates as described by Singh et al. (2006), except that liquid medium, rather than solidified agar medium, was used. Twenty microliter droplets of crude plant leaf extracts and the positive control were placed in the tributyrin/phenol red medium containing tributyrin, olive oil or Tween 20 and incubated at 30 C overnight. In these assays, only the porcine lipase positive control and plant tissue inoculated with pIPG745, pIPG746 and pIPG768 (all with the cloned bovine PGE) exhibited the color change from red to bright yellow in the indicator tubes (refer FIG. 2 for assay details). Both the porcine lipase and the transiently expressed bovine PGE utilized all three substrates: tributyrin, olive oil and Tween 20. Transient expression of bovine PGE was confirmed in tobacco, pepper, tomato, citrus, geranium, and rice plants. These plant assays demonstrated that the bovine PGE gene cloned in Example 6 and operationally constructed for plant expression in Example 12 was expressed in all plants tested, whether monocot or dicot, in an enzymatically active form.

### Example 21

### Use of Transient Expression Assays in Plants to Confirm Plant Expression of an Active, Correctly Folded Nematode Triglyceride Lipase-Cholesterol Esterase

For transient expression assays, the plant transformation and expression vectors constructed in Example 15 were moved into A. tumefaciens strain GV2260 by electroporation. GV2260 carrying pIPG823 was used for transient expression in tobacco and geranium plants exactly as described in Example 20. The supernatants from crude plant leaf extracts were directly used in phenol red lipase/esterase assays using tributyrin and olive oil as substrates exactly as described in Example 20. In these assays, only the porcine lipase positive control and plant tissue inoculated with pIPG823 (cloned nematode triglyceride lipase-cholesterol esterase) exhibited the color change from red to bright yellow in the indicator tubes (refer FIG. 2). Both the porcine lipase and the transiently expressed nematode triglyceride lipase-cholesterol esterase utilized tributyrin and olive oil. These plant assays further demonstrated that active esterase cloned from any source and operationally constructed for plant expression can be expressed in all plants tested in an enzymatically active form, without evident injury to the plant.

### Example 22

### Use of Bovine PGE as a Selectable Marker and Tween 20 as a Selective Agent to Select Transgenic Tomato Cells and Regenerate Transgenic Whole Plants

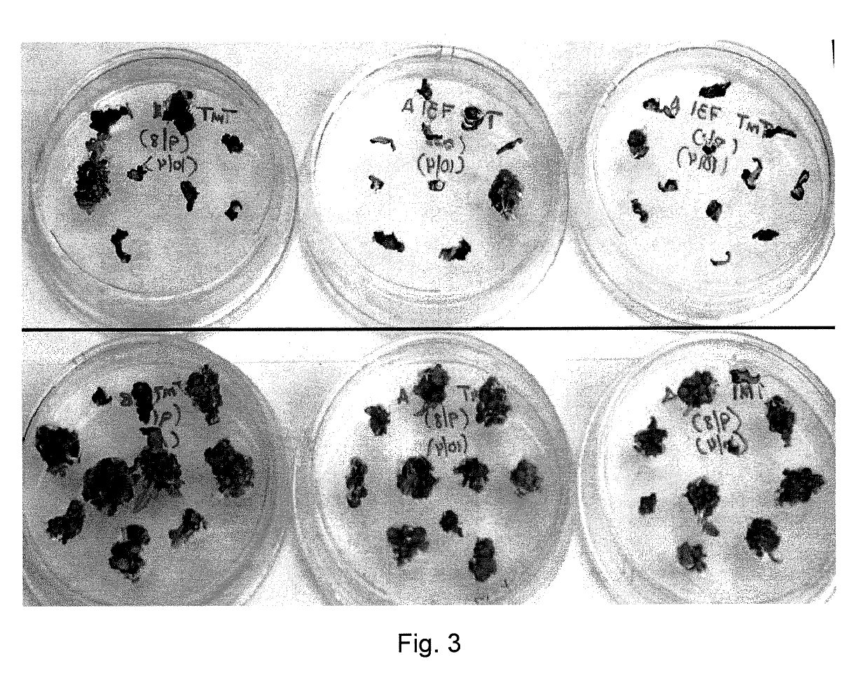

Transgenic tomato (Lycopersicon esculentum) cultivar “Micro-Tom” plants were created using the Agrobacterium tumefaciens DNA delivery method [Riggs et al., 2001; Sun et al., 2006] using pIPG768. Selection was either on 1% Tween 20 added to both shoot and root regeneration media. As controls for comparative purposes, 100 micrograms/ml kanamycin was used. After 30 days of selection on tomato regeneration medium supplemented with 1% Tween 20, nearly all of the tomato leaf explants provided with bovine PGE on pIPG768 produced abundant, primarily green callus that had some areas of brown necrotic tissue, while all of the tomato leaf material provided with empty vector control plasmid either died or produced a small amount of brown callus that died (FIG. 3). Furthermore, nearly half of the tomato leaf material provided with bovine PGE on pIPG768 produced green shoots on tomato regeneration medium supplemented with 1% Tween 20, while all of the tomato leaf material provided with empty vector control plasmid either produced no shoots or only small, aborted shoot primordia on 1% Tween 20 (FIG. 3). This result indicated that chimeric, transformed callus cells from the pIPG768 treated material were rescuing some nontransformed cells, but that shoots were formed only from transformed material. When placed on rooting medium with 1% Tween 20, the shoots readily rooted, and any remaining callus that was transferred along with the shoots died.

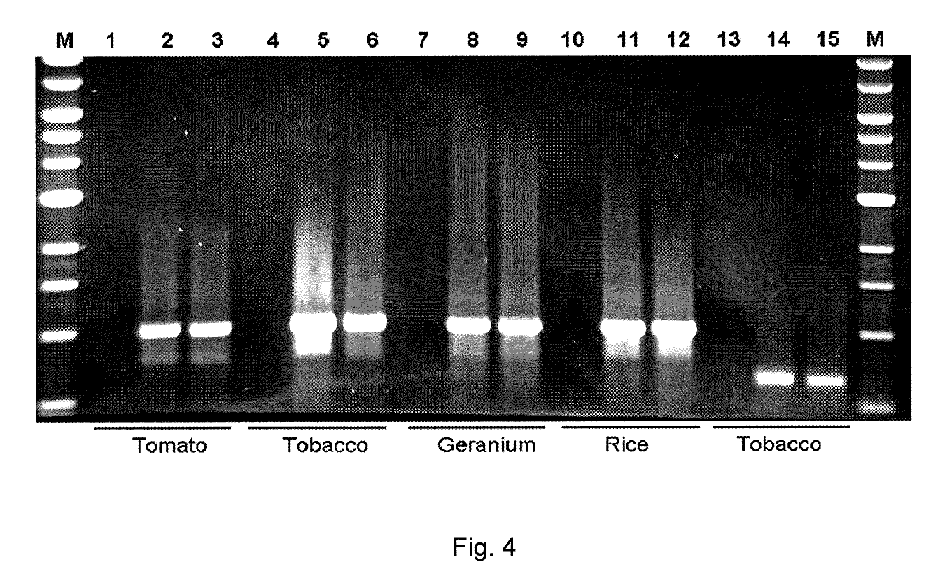

TABLE 4
Transformation frequencies of tomato cultivar Micro-Tom using
pIPG768 and 1% Tween 20 or 100 micrograms/ml kanamycin
added to both the shoot and root regeneration media for selection.
Transformation frequencies reported were confirmed by PCR
(refer FIG. 4).

| | | | No. of | | |
|---|---|---|---|---|---|
| Transgene on | | | independent | No. of | |
| pIPG768 | Exp. | No. of | calli forming | rooted | Transformation |
| selected | No. | explants | shoots | shoots | frequency (%) |
| Bovine PGE/ | 1 | 253 | 25 | 25 | 7% |
| 1% Tween | | | | | |
| Bovine PGE/ | 2 | 204 | 15 | 15 | 4% |
| 1% Tween | | | | | |
| Bovine PGE/ | 3 | 275 | 48 | 48 | 4% |
| 1% Tween | | | | | |
| nptII/ | 1 | 98 | 40 | 40 | 1% |
| Kanamycin | | | | | |

Transformation efficiency using pIPG768 and identical tissue culture methods ranged from 4% to 7% in these experiments when 1% Tween 20 was used for selection, as compared with 1% when 100 micrograms/ml kanamycin was used. Transformation was confirmed by PCR (FIG. 4) and also by the phenol red lipase/esterase agar assay (refer FIG. 2) described above in Example 20 for both 1% Tween 20 and kanamycin selection methods.

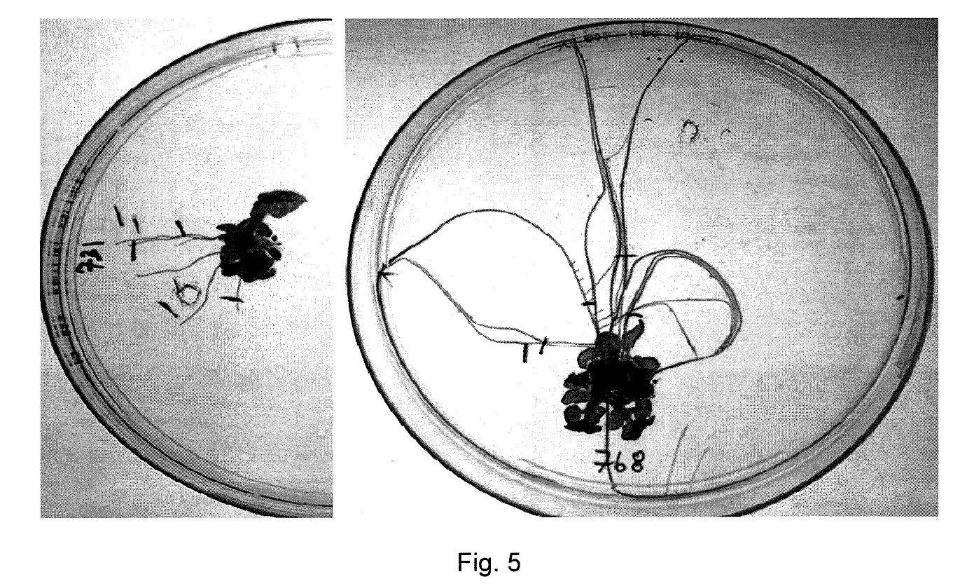

In order to test if the rooting medium inhibited or killed nontransgenic tomato that were rooting, regenerating nontransgenic tomato plants that were rooting, as well as transgenic tomato plants carrying pIPG678 that were rooting were placed on 1% Tween 20 medium. Tween 20 (1%) completely and almost immediately inhibited root growth of the nontransgenic plants, but did not affect growth of the transgenic plants expressing bovine PGE (FIG. 5).

### Example 23

### Use of Bovine PGE as a Selectable Marker and Tween 20 as a Selective Agent to Select Transgenic Tobacco Cells and Regenerate Transgenic Whole Plants

Transgenic tobacco (Nicotiana tabacum) cultivar “Xanthi” plants were created using the Agrobacterium tumefaciens DNA delivery method [Horsch et al., 1985] using pIPG833. Selection was on 1% or on 0.05% Tween 20 added to both shoot and root regeneration media. As a control for comparative purposes, pIPG786 was used (carrying the nptII gene for kanamycin resistance) for transformation and 100 micrograms/ml kanamycin was used for selection.

After 30 days of selection on tobacco regeneration medium supplemented with 1% Tween 20, nearly all of the tobacco leaf explants provided with bovine PGE on pIPG833 produced abundant, primarily green callus that had some areas of brown necrotic tissue, while all of the tobacco leaf material provided with empty vector control plasmid either died or produced a small amount of brown callus that died, similar to the tomato pieces in FIG. 3. However, only 16% of the tobacco leaf material provided with bovine PGE on pIPG833 produced green shoots on regeneration medium supplemented with 1% Tween 20, while all of the tobacco leaf material provided with empty vector control plasmid either produced no shoots or only small, aborted shoot primordia on 1% Tween 20, similar to the tomato explant tissue in FIG. 3. On 0.05% Tween, selection was much less efficient, and 100% of the tissue provided with pIPG833 produced shoots. In both cases, when placed on rooting medium with 0.05% or 1% Tween 20, the shoots readily rooted, but a much higher number of escapes were noticed on 0.05% Tween.

TABLE 5
Transformation frequencies of tobacco cultivar Xanthi using
pIPG833 and 1% Tween 20 or 0.05% Tween 20 added to
both the shoot and root regeneration media for selection, and
using pIPG786 as a control, using the nptII gene for selection
and 100 micrograms/ml kanamycin added to both the shoot
and root regeneration media. Transformation frequencies
reported were confirmed by PCR (refer FIG. 4).

| Bovine PGE on | | | | | |
|---|---|---|---|---|---|
| pIPG833 and | | | No. of | | |
| nptII on | | | independent | No. of | |
| pIPG786 | Exp. | No. of | calli forming | rooted | Transformation |
| selected | No. | explants | shoots | shoots | frequency (%) |
| Bovine PGE/ | 1 | 73 | 12 | 12 | 5% |
| 1% Tween | | | | | |
| Bovine PGE/ | 1 | 73 | 73 | 73 | 6% |
| 0.5% Tween | | | | | |
| nptII/100 | 1 | 235 | 55 | 55 | 19% |
| micrograms/ml | | | | | |
| kanamycin | | | | | |

Transformation efficiency using pIPG833 and identical tissue culture methods ranged from 5% to 6% in these experiments when Tween 20 was used for selection at the 1% or the 0.05% levels, respectively. Transformation using the nptII resistance gene on kanamycin containing media was more efficient than transformation using the bovine PGE gene on Tween 20 in these experiments. All transformations were confirmed by PCR (FIG. 4) and for pIPG833, also by the phenol red lipase/esterase agar assay (refer FIG. 2) described above in Example 20.

### Example 24

### Use of Bovine PGE as a Selectable Marker and Span 20 as a Selective Agent to Select Transgenic Tobacco Cells and Regenerate Transgenic Whole Plants

Transgenic tobacco (Nicotiana tabacum) cultivar “Xanthi” plants were created using the Agrobacterium tumefaciens DNA delivery method [Horsch et al., 1985] using pIPG833. Selection was on 1% or on 0.05% Span 20 added to both shoot and root regeneration media. After 30 days of selection on tobacco regeneration medium supplemented with 1% Span 20, nearly all of the tobacco leaf explants provided with bovine PGE on pIPG833 produced abundant, primarily green callus that had some areas of brown necrotic tissue, while all of the tobacco leaf material provided with empty vector control plasmid either died or produced a small amount of brown callus that died, similar to the tomato pieces in FIG. 3. However, only 17% of the tobacco leaf material provided with bovine PGE on pIPG833 produced green shoots on regeneration medium supplemented with 1% Span 20, while all of the tobacco leaf material provided with empty vector control plasmid either produced no shoots or only small, aborted shoot primordia on 1% Span 20, similar to the tomato explant tissue in FIG. 3. On 0.05% Span 20, selection was somewhat less efficient, and 41% of the tissue provided with pIPG833 produced shoots. In both cases, when placed on rooting medium with 0.05% or 1% Span 20, the shoots readily rooted, but a higher number of escapes were noticed on 0.05% Span 20.

TABLE 6
Transformation frequencies of tobacco cultivar Xanthi using
pIPG833 and 1% Span 20 or 0.05% Span 20 added to both
the shoot and root regeneration media for selection, and using
pIPG786 as a control, using the nptII gene for selection and
100 micrograms/ml kanamycin added to both the shoot and root
regeneration media. Transformation frequencies reported were
confirmed by PCR (refer FIG. 4).

| Bovine PGE on | | | | | |
|---|---|---|---|---|---|
| pIPG833 and | | | No. of | | |
| nptII on | | | independent | No. of | |
| pIPG786 | Exp. | No. of | calli forming | rooted | Transformation |
| selected | No. | explants | shoots | shoots | frequency (%) |
| Bovine PGE/ | 1 | 75 | 13 | 13 | 8% |
| 1% Span 20 | | | | | |
| Bovine PGE/ | 1 | 70 | 29 | 28 | 12% |
| 0.5% Span 20 | | | | | |
| nptII/100 | 1 | 235 | 55 | 55 | 19% |
| micrograms/ml | | | | | |
| kanamycin | | | | | |

Transformation efficiency using pIPG833 and identical tissue culture methods ranged from 8% to 12% in these experiments when Span 20 was used for selection at the 1% or the 0.05% levels, respectively. Span 20 appears to be more efficient for tobacco transformation than Tween 20 (compare data in Table 5 with Table 6), with efficiency using Span 20 in this example approaching that of kanamycin selection of the nptII gene. Transformation was confirmed by PCR (FIG. 4) and also by the phenol red lipase/esterase agar assay (refer FIG. 2) described above in Example 20.

### Example 25

### Use of Nematode Lipase as a Selectable Marker and Span 20 as a Selective Agent to Select Transgenic Tobacco Cells and Regenerate Transgenic Whole Plants

Transgenic tobacco (Nicotiana tabacum) cultivar “Xanthi” plants were created using the Agrobacterium tumefaciens DNA delivery method [Horsch et al., 1985] using pIPG875. Selection was on 1% Span 20 added to the shoot regeneration medium and 0.05% Span 20 added to the root regeneration medium. After 30 days of selection on tobacco shoot regeneration medium supplemented with 1% Span 20, nearly all of the tobacco leaf explants provided with bovine PGE on pIPG875 produced abundant, primarily green callus that had some areas of brown necrotic tissue, while all of the tobacco leaf material provided with empty vector control plasmid either died or produced a small amount of brown callus that died, similar to the tomato pieces in FIG. 3. On 0.05% Span 20 on rooting medium, selection was highly efficient as compared with the bovine PGE gene, and more than half of the shoots died; there were almost no “escapes” using this nematode lipase gene on pIPG875.

TABLE 7
Transformation frequencies of tobacco cultivar Xanthi using
pIPG875 and 1% Span 20 added to shoot regeneration medium
and 0.05% Span 20 added for root regeneration medium for
selection. pIPG786 is shown as a comparative control, using the
nptII gene for selection and 100 micrograms/ml kanamycin added
to both the shoot and root regeneration media. Transformation
frequencies reported were confirmed by PCR (refer FIG. 4).

| Nematode | | | | | |
|---|---|---|---|---|---|
| lipase | | | | | |
| on pIPG833 | | | No. of | | |
| and nptII on | | | independent | No. of | |
| pIPG786 | Exp. | No. of | calli forming | rooted | Transformation |
| selected | No. | explants | shoots | shoots | frequency (%) |
| Nematode | 1 | 50 | 23 | 10 | 39% |
| lipase/1% | | | | | |
| Span 20 | | | | | |
| nptII/100 | 1 | 235 | 55 | 55 | 19% |
| micrograms/ml | | | | | |
| kanamycin | | | | | |

Transformation efficiency using pIPG875 and identical tissue culture methods to used on tobacco in the other Examples, above, was 39%, in this experiments. In this example, nematode lipase was more efficient as a selectable marker on pIPG875 than either bovine PGE on pIPG833 or the nptII gene on pIPG786. This example demonstrates that the specific esterase gene used for selection may make a significant difference in efficiency of selection.

### Example 26

### Use of Bovine PGE as a Selectable Marker and Span 20 as a Selective Agent to Select Transgenic Geranium Cells and Regenerate Transgenic Whole Plants

Transgenic geranium (Pelargonium X hortorum) cv. “Avenida” plants were created using the Agrobacterium tumefaciens DNA delivery method [Riggs et al., 2001; Sun et al., 2006] using pIPG833. Selection of leaf petiole explants was on 1%, 0.5% and 0.25% Span 20 added to shoot regeneration medium; beginning after three weeks, any shoots that emerged were excised from the petiole piece and transferred to root regeneration medium supplemented with Span at one half the concentration used in the shoot regeneration medium. As a control for comparative purposes, pIPG835 was used (carrying the hygromycin antibiotic resistance gene) for transformation and 10 micrograms/ml hygromycin was used for selection. Span 20 at 1% concentration killed 35% of the leaf petioles after three weeks, and 100% of the petioles after 10 weeks, and no shoots emerged. Span 20 at 0.5% concentration was highly efficient for geranium transformation, allowing no escapes and giving the same confirmed transformation frequency as Span 20 at 0.25%.

TABLE 8
Transformation frequencies of geranium (Pelargonium X hortorum)
cv. Avenida using pIPG833 to provide the selectable esterase
marker and Span 20 at the indicated concentrations used as the selection
agent in shoot regeneration medium. Half the indicated concentration of
Span 20 was used in the root regeneration medium. pIPG835 was used as a
control to provide the selectable hygromycin resistance gene marker and
hygromycin at 10 micrograms/ml added to both the shoot and root
regeneration media. Transformation frequencies reported were confirmed
by PCR (refer FIG. 4).

| Bovine PGE on pIPG833 | | | No. of independent | No. of | |
|---|---|---|---|---|---|
| and hygromycin resistance | Exp | No. of | petiole explants | rooted | Transformation |
| on pIPG835 selected | No. | explants | forming shoots | shoots | frequency (%) |
| Bovine PGE/1% Span 20 | 1 | 160 | 0 | 0 | 0% |
| Bovine PGE/0.5% Span 20 | 1 | 160 | 6 | 6 | 4% |
| Bovine PGE/0.25% Span | 1 | 160 | 33 | 33 | 4% |
| 20 | | | | | |
| hygromycin resistance/ | 1 | 442 | 115 | 40 | 6% |
| 10 micrograms/ml | | | | | |
| hygromycin | | | | | |

Transformation efficiencies using pIPG833 on Span 20 for selection and pIPG835 as a control using hygromycin for selection were very similar, at 4% to 6%, using identical tissue culture methods. Span 20 at 0.5% in the shooting medium and 0.25% in rooting medium appears to be more efficient for geranium transformation than Span 20 at 0.25% in the shooting medium and 0.125% in the rooting medium, since a large number of nontransgenic “escape” plants resulted from using this lower level of Span 20 (81%). Use of hygromycin at 10 micrograms/ml resulted in regeneration of 27% nontransgenic escape plants. Transformation was confirmed by PCR (FIG. 4) for all transformants and also for a limited number of transgenic geranium plants by the phenol red lipase/esterase agar assay (refer FIG. 2) described above in Example 20.

### Example 27

### Use of Bovine PGE as a Selectable Marker and Span 20 as a Selective Agent to Select Transgenic Rice Cells and Regenerate Transgenic Whole Plants

Transgenic rice (Oryza sativa japonica var. TP-309) plants were created using the Agrobacterium tumefaciens DNA delivery method and pIPG833, using callus produced from seeds (Hiei et al., 1997). Selection of was on 1% and 0.5% Span 20 added to shoot regeneration medium; any shoots that emerged were excised from the callus and transferred to root regeneration medium supplemented with Span 20 at one half the concentration used in the shoot regeneration medium. Span 20 at 1% concentration killed over 90% of the callus pieces after 15 weeks and since no shoots emerged, the experiment was terminated. However, three shoots out of 60 rice seeds initially placed into tissue culture emerged on Span 20 and two of these were positive for bovine PGE.

TABLE 9
Transformation frequency of rice var. TP-309 using
pIPG833 to provide the selectable esterase marker and Span 20 at the
indicated concentrations used as the selection agent in shoot regeneration
medium. Half the indicated concentration of Span 20 was used in the root
regeneration medium. Transformation frequencies reported were confirmed
by PCR (refer FIG. 4).

| | | | No. of independent | No. of | |
|---|---|---|---|---|---|
| Bovine PGE on pIPG833 | Exp | No. of | petiole explants | rooted | Transformation |
| selected | No. | explants | forming shoots | shoots | frequency (%) |
| Bovine PGE/1% Span 20 | 1 | 60 | 0 | 0 | 0% |
| Bovine PGE/0.5% Span 20 | 1 | 60 | 3 | 2 | 3% |

Transformation efficiencies using pIPG833 on Span 20 for selection at 0.5% in the shooting medium and 0.25% in rooting medium appears to be reasonably efficient for this species and similar to that found for geranium. Transformation was confirmed by PCR (FIG. 4) for all transformants. This example extends the utility of the use of esterases for selection in plant tissue culture medium to include the monocot rice.

### Example 28

### Use of Bovine PGE as a Selectable Marker and Span 20 as a Selective Agent to Select Transgenic Sugarcane Cells and Regenerate Transgenic Whole Plants

Transgenic sugarcane belonging to the genus Saccharum (Poaceae) and including a variety of different species are created using either biolistic bombardment or the Agrobacterium tumefaciens DNA delivery method and pIPG833. A variety of different methods may be used (reviewed by Lakshmanan et al., 2005, 2006a, 2006b). A preferred method is by Manickavasagam et al. (2004) using A. tumefaciens. Selection is on 0.5% Span 20 added to shoot regeneration medium; any shoots that emerge are excised from the callus and transferred to root regeneration medium supplemented with Span 20 at one half the concentration used in the shoot regeneration medium.

### Example 29

### Sexual Reproduction of Transgenic Plants Expressing PGE Proteins

Transgenic diploid tomato, rice and tobacco plants were obtained as set forth in Examples 22, 23, 24, 25 and 27, wherein the transgenic plants expressed the introduced nucleic acid molecule coding for an esterase. The transgenic (T₀ generation) tomato, rice and tobacco plants were self-pollinated and the seed (T₁ generation) was harvested from the self-pollinated plants, processed, planted, and progeny plants grown from the self-pollinated-seed. PCR assays were used to determine that the T₁ progeny plants all had a classical genetic 3:1 ratio, wherein ¾ of the plants (¼ homozygous transgenic and ½ heterozygous transgenic plants) were found to be transgenic by PCR tests, and ¼ of the plants were nontransgenic. These tests showed that that the introduced nucleic acid molecules coding for the PGE proteins were stably integrated into tomato using the methods of the present invention and that such nucleic acid molecules are heritable.

### Example 30

### Asexual Reproduction of Transgenic Plants Expressing PGE

Transgenic geranium, tomato and tobacco plants were obtained as set forth in Examples 22, 23, 24, 25 and 26, wherein the transgenic plants expressed the introduced nucleic acid molecule coding for an esterase. The transgenic geranium and tomato plants were asexually propagated to produce progeny clones using techniques well known to one skilled in the art of geranium, tobacco or tomato propagation. For geranium and other vegetative species that are normally propagated by taking cuttings, an internode with two nodes are cut from a mother plant and rooted, normally using a support medium, with or without root inducing hormones, producing a single new plant for each such clone or “cutting”. The cuttings were in all cases genetically identical to the mother plant; the genetic modifications performed in the mother plant were stable through at least two generations, as indicated by PCR and the phenol red lipase/esterase tests of Example 20.

It must be noted that as used in this specification and the appended claims, the singular forms “a,” “and,” and “the” include plural referents unless the contexts clearly dictates otherwise. Thus, for example, reference to “a lipase” includes any one, two, or more of the lipases encoded by genes in at least 35 different families; reference to “a transgenic plant” includes large numbers of transgenic plants and mixtures thereof, and reference to “the method” includes one or more methods or steps of the type described herein.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications cited herein are incorporated herein by reference for the purpose of disclosing and describing specific aspects of the invention for which the publication is cited.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### LITERATURE CITED

Abd Maurad, Z., R. Ghazali, P. Siwayanan, Z. Ismail, and S. Ahmad. 2006. Alpha-sulfonated methyl ester as an active ingredient in palm-based powder detergents. Journal of Surfactants and Detergents 9, no. 2:161-167.
Arce P et al. 1999. Enhanced resistance to bacterial infection by Erwinia carotovora subsp. Atroseptica in transgenic potato plants by expressing the attacin or the cecropin SB-37 genes. American Journal of Potato Research 76:169-177.
Broekaert, W. F. et al. 1997. Antimicrobial peptides from plants. Critical Reviews in Plant Sciences 16:297-323
Callis, J., M. Fromm, and V. Walbot. 1987. Introns increase gene-expression in cultured maize cells. Genes & Development 1:1183-1200.
Calvo, M. V. and Fontecha, J. 2004. Purification and characterization of a pregastric esterase from a hygienized kid rennet paste. J. Dairy Sci. 87:1132-1142.
Ceccardi, T. L., G. A. Barthe, and K. S. Derrick. 1998. A novel protein associated with citrus blight has sequence similarities to expansin. Plant Molecular Biology 38:775-783.
Claffey, D. J., J. D. Meyer, R. Beauvais, T. Brandt, E. Shefter, D. J. Kroll, J. A. Ruth, and M. C. Manning. 2000. Long chain arginine esters: A new class of cationic detergents for preparation of hydrophobic ion-paired complexes. Biochemistry and Cell Biology-Biochimie et Biologie Cellulaire 78, no. 1:59-65.
Curtis, I. S. and H. G. Nam. 2001. Transgenic radish (Raphanus sativus L. longipinnatus Bailey) by floral-dip method—plant development and surfactant are important in optimizing transformation efficiency. Transgenic Research 10, no. 4:363-371.
Desnuelle, P. and Savary, P. 1963. Specificity of lipases. J. Lipid Research 4:309-384.
Duan Y P et al. 1999. Expression of a single, host-specific gene in citrus cells elicits division, enlargement and cell death. Molecular Plant-Microbe Interactions 12:556-560
During K et al. 1993. Transgenic potato plants resistant to the phytopathogenic bacterium Erwinia carotovora. Plant J 3:587-598
During, K., P. Porsch, A. Mahn, O. Brinkmann, and W. Gieffers. 1999. The non-enzymatic microbicidal activity of lysozymes. FEBS Letters 449:93-100.
Gruber, V., Berna, B. P., Arnaud, T., et al. 2001. Large-scale production of a therapeutic protein in transgenic tobacco plants: effect of subcellular targeting on quality of a recombinant dog gastric lipase. Molec. Breeding 7:329-340.
Gupta, R., Rathi, P., Gupta, N., Bradoo, S. 2003. Lipase assays for conventional and molecular screening: an overview. Biotechnol. Appl. Biochem. 37:63-71.
Heeb, S., Itoh, Y., Nishijyo, T., Schnider, U., Keel, C., Wade, J., Walsh, U., O'Gara, F. and Haas, D. 2000. Small, stable shuttle vectors based on the minimal pVS1 replicon for use in gram-negative, plant-associated bacteria. Mol. Plant. Microbe Interact. 13: 232-237.
Hiei Y, Komari T, Kubo T., 1997. Transformation of rice mediated by Agrobacterium tumefaciens. Plant Mol. Biol. 35:205-18.
Horsch R. B., Fry J. E., Hoffmann N. L., Eichholtz D., Rogers S. G. and Fraley R. T. 1985. A simple and general method for transferring genes into plants. Science 227: 1229-1231.
Ibrahim, H. R., Thomas, U., and Pellegrini, A. 2001. A helix-loop-helix peptide at the upper lip of the active site cleft of lysozyme confers potent antimicrobial activity with membrane permeabilization action. J. Biol. Chem. 276:43767-43774.
Jaynes J M et al. 1987. Increasing bacterial disease resistance in plants utilizing antibacterial genes from insects. Bioassays 6:263-270
Jaeger, K. E. and Reetz, M. T. 1998. Microbial lipases form versatile tools for biotechnology. Trends Biotechnol. 16:396-403.
Jette, J. F. and Ziomek, E. 1994. Determination of lipase activity by a Rhodamine-Triglyceride-Agarose assay. Analytical Biochemistry 219:256-260.
Kapila, J., R. De Rycke, M. Van Montagu, and G. Angenon. 1997. An Agrobacterium-mediated transient gene expression system for intact leaves. Plant Science 122:101-108.
Kato, A., S. Nakamura, H. Ibrahim, T. Matsumi, C. Tsumiyama, and M. Kato. 1998. Production of genetically modified lysozymes having extreme heat stability and antimicrobial activity against Gram negative bacteria in yeast and in plant. Nahrung-Food 42:128-130
Kim, J. C., Choi, G. J., Lee, S. W., Kim, J. S., Chung, K. Y., and Cho, K. Y. 2004. Screening extracts of Achyranthes japonica and Rumex crispus for activity against various plant pathogenic fungi and control of powdery mildew. Pest Management Science 60:803-808.
Kingsley, M. T., D. W. Gabriel, G. C. Marlow, and P. D. Roberts. 1993. The opsX locus of Xanthomonas campestris affects host range and biosynthesis of lipopolysaccharide and extracellular polysaccharide. J. Bacteriol. 175:5839-5850.
Ko K. 1999. Attacin and T4 lysozyme transgenic in Galaxy apple: Regulation of transgene expression and plant resistance to fire blight (Erwinia amylovora). PhD dissertation, Cornell University, Ithaca N.Y. 194 pp.
Ko K et al. 2000. Effect of untranslated leader sequence of AMV RNA 4 and signal peptide of pathogenesis-related protein 1b on attacin gene expression, and resistance to fire blight in transgenic apple. Biotechnology Letters 22:373-381.
Lakshmanan, P., Geijskes, R. J., Wang, L. F., Elliott, A., Grof, C. P. L., Berding, N., and Smith, G. R. 2006a. Developmental and hormonal regulation of direct shoot organogenesis and somatic embryogenesis in sugarcane (Saccharum spp. interspecific hybrids) leaf culture. Plant Cell Reports 25:1007-1015.
Lakshmanan, P. 2006b. Somatic embryogenesis in sugarcane—An addendum to the invited review ‘Sugarcane biotechnology: The challenges and opportunities,’ in vitro cell. Dev. Biol. Plant 41(4): 345-363; 2005. In Vitro Cellular & Developmental Biology-Plant 42:201-205.
Lakshmanan, P., Geijskes, R. J., Aitken, K. S., Grof, C. L. P., Bonnett, G. D., and Smith, G. R. 2005. Sugarcane biotechnology: The challenges and opportunities. In Vitro Cellular & Developmental Biology-Plant 41:345-363.
Li Q et al. 2001. Enhanced disease resistance conferred by expression of an antimicrobial magainin analog in transgenic tobacco. Planta 212:635-639
Manickavasagam, M., Ganaphthi, A., Anbazhagan, V. R., Sudhakar, B., Selvaraj, N., Vasudevan, A., and Kasthurirengan, S. 2004. Agrobacterium-mediated genetic transformation and development of herbicide-resistant sugarcane (Saccharum species hybrids) using axillary buds. Plant Cell Reports 23:134-143.
Mitra A and Zhang Z. 1994. Expression of a human lactoferrin cDNA in tobacco cells produces antibacterial protein(s). Plant Physiol 106:977-981.
Moore G. A., Jacono, C. C., Neidigh J. L., Lawrence S. D. and Cline K., 1992. Agrobacterium-mediated transformation of citrus stem segments and regeneration of transgenic plants. Plant Cell Rep 11:238-242.
Mun, J. H., Lee, S. Y., Yu, H. J., Jeong, Y. M., Shin, M. Y., Kim, H., Lee, I., and Kim, S. G. Petunia actin-depolymerizing factor is mainly accumulated in vascular tissue and its expression is enhanced by the first intron. Gene 292, 233-243. 2004.
Munford, R. S, and Hunter, J. P. 1992. Acyloxyacyl hydrolase, a leukocyte enzyme that deacylates bacterial lipopolysaccharides, has phospholipase, lysophospholipase, diacylglycerollipase, and acyltransferase activities in vitro. J. Biol. Chem. 267: 10116-10121.
Nakajima H et al. 1997. Fungal and bacterial disease resistance in transgenic plants expressing human lysozyme. Plant Cell Rep 16:674-679.
Norelli J L et al 1994. Transgenic Mailing 26 apple expressing the attacin E gene has increased resistance to Erwinia amylovora. Euphytica 77:123-128.
Norelli J L et al. 1998. Effect of cercropin-type transgenes on fire blight resistance of apple. Acta Hort 489:273-278.
Norelli J L et al. 1999. Genetic transformation for fire blight resistance in apple. Acta Hort 489:295-296.
Owens, L. D. and Heutte, T. M. (1997) A single amino acid substitution in the antimicrobial defense protein cecropin B is associated with diminished degradation by leaf intercellular fluid. Molecular Plant-Microbe Interactions. 10, 525-528.
Reddy, J D, Reddy, S L, Hopkins, D L, and Gabriel, D W. 2007. ToIC is required for pathogenicity of Xylella fastidiosa in Vitis vinifera grapevines. Molec. Plant-Microbe Interact. 20:403-410.
Reuveni, M., Agapov, V., and Reuveni, R. 1995. Suppression of Cucumber Powdery Mildew (Sphaerotheca-Fuliginea) by Foliar Sprays of Phosphate and Potassium-Salts. Plant Pathology 44:31-39.
Reynoird J P et al. 1999. First evidence for differences in fire blight resistance among transgenic pear clones expressing attacin gene. Plant Science 149:23-31.
Riggs, C. D., K. Zeman, R. DeGuzman, A. Rzepczyk and A. A. Taylor. 2001. Antisense inhibition of a tomato meiotic proteinase suggests functional redundancy of proteinases during microsporogenesis Genome 44: 644-650.
Robichon, M. P., J. P. Renou and R. Jalouzot, 1995. Genetic transformation of Pelargonium X hortorum. Plant Cell Reports 15:63-67.
Rose, A. B. and Beliakoff, J. A. 2004. Intron-mediated enhancement of gene expression independent of unique intron sequences and splicing. Plant Physiol. 122, 535-542.
Rose, A. B. 2002. Requirements for intron-mediated enhancement of gene expression in Arabidopsis. Rna-A Publication of the Rna Society 8:1444-1453
Roussel A, Miled N, Berti-Dupuis L, Riviere M, Spinelli S, Berna P, Gruber V, Verger R, Cambillau C. 2002. Crystal structure of the open form of dog gastric lipase in complex with a phosphonate inhibitor. J. Bio. Chem. 277: 2266-2274.
Sambrook, J., Fritsch, E. F. and Maniatis, T., 1989. Molecular Cloning: A Laboratory Manual, Cold Spring Harbor Laboratory Press, NY.
Simpson, G. G. and Filipowicz, W. Splicing of precursors to mRNA in higher plants: mechanism, regulation an sub-nuclear organization of the spliceosomal machinery. Plant Mol. Biol. 32, 1-41. 1996.
Singh, R., Gupta, N., Goswami, V. K. and R. Gupta. 2006. A simple activity staining protocol for lipases and esterases. Appl. Microbiol. Biotechnol. 70:679-682.
Sun, H. J., S. Uchii, S. Watanabe, and H. Ezura. 2006. A highly efficient transformation protocol for Micro-Tom, a model cultivar for tomato functional genomics. Plant and Cell Physiology 47, no. 3:426-431.
Taguchi S et al. 2000. Functional mapping against Escherichia coli for the broad-spectrum antimicrobial peptide, thanatin, based on an in vivo monitoring assay system. J Biochem 128:745-754.
Timmermans, M. Y. J., Teuchy, H., and Kupers, L P M. 1998. The cDNA sequence encoding bovine pregastric esterase. Gene 147: 259-262.
Trudel J et al. 1995. Secreted hen lysozyme in transgenic tobacco: Recovery of bound enzyme and in vitro growth inhibition of plant pathogens. Plant Science 106:55-62.
Vunnam S et al. 1997. Synthesis and antibacterial action of cecropin and proline-arginine-rich peptides from pig intestine. J Peptide Res 49:59-66.
Wang Y et al. 1999. Porcine pulmonary surfactant preparations contain the antibacterial peptide prophenin and a C-terminal 18-residue fragment thereof. FEBS Lett 460:257-262.
Wroblewski, T., Tomczak, A. and Michelmore, R. 2005. Optimization of Agrobacterium mediated transient assays of gene expression in lettuce, tomato and Arabidopsis. Plant Biotechnology J. 3:259-273.

### Sequence Listings

Sequence ID #1. DNA primer used to identify bovine PGE gene from transgenic plants, IPG952.

“CTCAGCTGCATACGCCTTCC”

Sequence ID #2. DNA primer used to identify bovine PGE gene from transgenic plants, IPG953.

“ACAGGTCATTGTCAGCACTCC”

Sequence ID #3. DNA primer used to amplify bovine PGE gene from calf salivary gland cDNA, IPG451.

“atgcccatggaacatatgatgtggtggctacttgtaaca”

Sequence ID #4. DNA primer used to amplify bovine PGE gene from calf salivary gland cDNA, IPG452.

“gcat cccggg cta gagctc ctttttgtcttcggccatcaa”

Sequence ID #5. Bovine pregastric esterase cloned by IPG, translation product, with native leader and two amino acids added to form an ER retention signal sequence.

“MWWLLVTVCFIHMSGNAFC FLGKIAKNPEASMNVSQMISYWGYPS EMHKVITADGYILQVYRIPHGKNNANHLGQRPVVFLQHGLLGSATNWISN LPKNSLGFLLADAGYDVWLGNSRGNTWAQEHLYYSPDSPEFWAFSFDEMA EYDLPSTIDFILRRTGQKKLHYVGHSQGTTIGFIAFSTNPTLAEKIKVFY ALAPVATVKYTKSLFNKLALIPHFLFKIIFGDKMFYPHTFLEQFLGVEMC SRETLDVLCKNALFAITGVDNKNFNMSRLDVYIAHNPAGTSVQNTLHWRQ AVKSGKFQAFDWGAPYQNLMHYHQPTPPIYNLTAMNVPIAVWSADNDLLA DPQDVDLLLSKLSNLIYHKEIPNYNHLDFIWAMDAPQEVYNEIVSLMAEDKKEL*”

Sequence ID #6. DNA primer used to amplify nematode triglyceride lipase-cholesterol esterase gene from cDNA, IPG915.

“CAGCTGCATACGCcgaaaatgtcaccactcc”

Sequence ID #7. DNA primer used to amplify nematode triglyceride lipase-cholesterol esterase gene from cDNA, IPG894.

“ttacgaaatagtatctggaag”

Sequence ID #8. Nematode triglyceride lipase-cholesterol esterase cloned by IPG, translation product.

“MKSLLLLFLLFLHTLCENVTTPNSEEDTDMTATPSTITPLLSTAQNPSLPK TSKLPLALKTPVPTFPFSSLATSDWLSSMPTIQTLLPPPLPLTTLEVPENENFGFTSLAPLWT LPTQPPAWSPMLDSPIKPIQNSMFPTFPTMPTLPTLPTLAPFTFPTLPPPTTMKPLNITIDPE ALMDVPEIITHWGYPVETHKVVTVDGYILTLHRIPHSKNETSKSASKTPKPVVFLQHGLLC TSSIWLLNLPRQSAGYIFADQGYDVWLGNMRGNTYSKEHTRMTSADRRFWKFSWEEM ARYDLPAMINYALKTTKRQNLYYVGHSQGALTMFAKMSEDPEMSKKIRKFFAMAPVAR MSHVKGLFQNLGQIYEQYNLVYQVFGDGEFLTNNIFTKLLTDIFCDQAVNNPLCENFIFA VSGPNSNQFNNSRIGIYLAHNPAGTSSRNILHFAQMVKKKRMSRFDHGKDLNLKIYGAP SPPEYDIRKINSSIYLFYSDFDWLANPKDVEGFLIPMLPSKTLKKATKLRDFNHNDFLWGM RARKEIYDKIINTIKLDQRRVKLQNSMERFFERQSRNPTSGLDEETMMRLRNETMNLD*”

Sequence ID #9. DNA primer used to amplify bacterial tributyrin esterase nematode gene from DNA, IPG922.

“ccATGGCAGTAATCAATATCGAA”

Sequence ID #10. DNA primer used to amplify bacterial tributyrin esterase nematode gene from DNA, IPG923.

“TATTAACTCAATCGTTCTTCTTGC”

Sequence ID #11. Bacterial tributyrin esterase cloned by IPG, translation product.

“mavinieyysevlgmnrkvnviypesskvedfsnteipvlyllhgmsgnenswmirs gierlirhtnlaivmpstdlgfyvnttygmnyfdaialelpkvihnffpnlstkkeknfiaglsmggygayrlal gtdhfsyaasisgvltfdgmeenfkenpaywggifgnwetfkgsdneilaladrkneerpklyawcgkqd flfpgneyaiaelkkkgfdvtyessdgvhewyywtkkiesvlqwlpinykqeerls*”

Sequence ID #12. DNA primer used to amplify plant carboxylesterase gene from cDNA, IPG970.

“TCatgagtatctccggtgctg”

Sequence ID #13. DNA primer used to amplify plant carboxylesterase gene from cDNA, IPG971.

“ACTAGTtcaaccttcgaggctgag”

Sequence ID #14. A. thaliana carboxylesterase cloned by IPG, translation product.

“MSISGAAVGSGRNLRRAVEFGKTHVVRPKGKHQATIVWLHGLGDNGSS WSQLLETLPLPNIKWICPTAPSQPISLFGGFPSTAWFDVVDINEDGPDDMEGLDVAAAHV ANLLSNEPADIKLGVGGFSMGAATSLYSATCFALGKYGNGNPYPINLSAIIGLSGWLPCA KTLAGKLEEEQIKNRAASLPIVVCHGKADDVVPFKFGEKSSQALLSNGFKKVTFKPYSAL GHHTIPQELDELCAWLTSTLSLEG*”

Sequence ID #15. DNA primer used to amplify plant lipase gene with SGNH motif from cDNA, IPG972.

“cccatggcttcttcactg”

Sequence ID #16. DNA primer used to amplify plant lipase gene with SGNH motif from cDNA, IPG973.

“ACTAGTccctttatgtatccactg”

Sequence ID #17. A. thaliana lipase with SGNH motif cloned by IPG, translation product.

“MASSLKKLISSFLLVLYSTTIIVASSESRCRRFKSIISFGDSIADTGNYLHLSD VNHLPQSAFLPYGESFFHPPSGRASNGRLIIDFIAEFLGLPYVPPYFGSQNVSFEQGINFAV YGATALDRAFLLGKGIESDFTNVSLSVQLDTFKQILPNLCASSTRDCKEMLGDSLILMGEI GGNDYNYPFFEGKSINEIKELVPLIVKAISSAIVDLIDLGGKTFLVPGGFPTGCSAAYLTLFQ TVAEKDQDPLTGCYPLLNEFGEHHNEQLKTELKRLQKFYPHVNIIYADYHNSLYRFYQEP AKYGFKNKPLAACCGVGGKYNFTIGKECGYEGVNYCQNPSEYVNWDGYHLTEAAYQK MTEGILNGPYATPAFDWSCLGSGTVDT*”

Sequence ID #18. Bovine pregastric esterase cloned by IPG with native leader replaced by the citrus P12 leader and two amino acids added to form an ER retention signal sequence, translation product

“MGVGTKVLVITTMAICLISSAAYA FLGKIAKNPEASMNVSQMISYWGYPS EMHKVITADGYILQVYRIPHGKNNANHLGQRPVVFLQHGLLGSATNWISN LPKNSLGFLLADAGYDVWLGNSRGNTWAQEHLYYSPDSPEFWAFSFDEMA EYDLPSTIDFILRRTGQKKLHYVGHSQGTTIGFIAFSTNPTLAEKIKVFY ALAPVATVKYTKSLFNKLALIPHFLFKIIFGDKMFYPHTFLEQFLGVEMC SRETLDVLCKNALFAITGVDNKNFNMSRLDVYIAHNPAGTSVQNTLHWRQ AVKSGKFQAFDWGAPYQNLMHYHQPTPPIYNLTAMNVPIAVWSADNDLLA DPQDVDLLLSKLSNLIYHKEIPNYNHLDFIWAMDAPQEVYNEIVSLMAEDKKEL*”

Sequence ID #1 g. Bovine pregastric esterase with P12 leader coding sequence and ER retention signal sequence added.

“ATGGGTGTAG GCACAAAAGT TCTGGTGATC ACAACTATGG CCATATGCCT AATTAGCTCA GCTGCATACG CCTTCCTTGG AAAAATTGCT AAGAACCCTG AAGCCAGTAT GAATGTTAGT CAGATGATTT CCTACTGGGG CTACCCAAGT GAGATGCATA AAGTTATAAC TGCGGATGGT TATATCCTTC AGGTCTATCG GATTCCTCAT GGAAAGAATA ATGCTAATCA TTTAGGTCAG AGACCTGTTG TGTTICTGCA GCATGGTCTT CTTGGATCAG CCACAAACTG GATTTCCAAC CTGCCCAAGA ACAGCCTGGG CTTCCTCCTG GCAGATGCTG GTTATGACGT GTGGCTGGGG AACAGCAGAG GAAACACCTG GGCCCAGGAA CATTTATACT ATTCACCAGA CTCCCCGGAA TTCTGGGCTT TCAGCTTTGA TGAAATGGCT GAATATGACC TTCCATCTAC AATTGATTTC ATCTTAAGGA GAACAGGACA GAAGAAGCTA CACTATGTTG GCCATTCCCA AGGCACCACC ATTGGTTTTA TCGCCTTTTC TACCAATCCC ACATTGGCTG AAAAAATCAA AGTCTTCTAT GCATTAGCCC CAGTTGCCAC AGTGAAGTAC ACCAAGAGCC TGTTTAACAA ACTTGCACTT ATTCCTCACT TCCTCTTCAA GATTATATTT GGTGACAAAA TGTTCTACCC ACACACTT TTTTGGAACAAT TTCTTGGTGT TGAAATGTGC TCCCGTGAGA CACTGGATGT CCTTTGTAAG AATGCCTTGT TTGCCATTAC TGGAGTTGAC AATAAAAACT TCAACATGAG TCGCTTAGAT GTGTATATAG CACATAATCC AGCAGGAACT TCTGTTCAAA ACACCCTCCA CTGGAGACAG GCTGTTAAGT CTGGGAAATT CCAAGCTTTT GACTGGGGAG CCCCATATCA GAACCTAATG CATTATCATC AGCCCACACC TCCCATCTAC AATTFAACAG CCATGAATGT CCCAATTGCA GTATGGAGTG CTGACAATGA CCTGTTGGCT GACCCTCAGG ATGTTGACCT TCTGCTTTCA AAACTCTCTA ATCTCATTTA CCACAAGGAA ATTCCAAATT ACAATCACTT GGACTTTATC TGGGCAATGG ATGCACCTCA AGAAGTTTAC AATGAAATTG TTTCTTTGAT GGCCGAAGAC AAAAAGGAGC TCTAG”

Sequence ID #20. DNA primer used to amplify and reclone bovine PGE gene and remove ER retention signal, IPG929.

“cgaacggctgttaagtctgggaa”

Sequence ID #21. DNA primer used to amplify and reclone bovine PGE gene and remove ER retention signal, IPG928.

“ccaactagtattactttttgtcttcggccatc”

Sequence ID #22. Bovine pregastric esterase with native leader replaced by the citrus P12 leader and without ER retention signal, translation product.

“MGVGTKVLVITTMAICLISSAAYAFLGKIAKNPEASMNVSQMISYWGYPS EMHKVITADGYILQVYRIPHGKNNANHLGQRPVVFLQHGLLGSATNWISNLPKNSLGFL LADAGYDVWLGNSRGNTWAQEHLYYSPDSPEFWAFSFDEMAEYDLPSTIDFILRRTGQK KLHYVGHSQGTTIGFIAFSTNPTLAEKIKVFYALAPVATVKYTKSLFNKLALIPHFLFKIIFG DKMFYPHTFLEQFLGVEMCSRETLDVLCKNALFAITGVDNKNFNMSRLDVYIAHNPAG TSVQNTLHWRQAVKSGKFQAFDWGAPYQNLMHYHQPTPPIYNLTAMNVPIAVWSAD NDLLADPQDVDLLLSKLSNLIYHKEIPNYNHLDFIWAMDAPQEVYNEIVSLMAEDKK*”

Sequence ID #23. Nematode triglyceride lipase-cholesterol esterase cloned by IPG, translation product, with native leader replaced by the citrus P12 leader, translation product.

“MGVGTKVLVITTMAICLISSAAYAENVTTPNSEEDTDMTATPSTITPLLS TAQNPSLPKTSKLPLALKTPVPTFPFSSLATSDWLSSMPTIQTLLPPPLPLTTLEVPENENF GFTSLAPLWTLPTQPPAWSPMLDSPIKPIQNSMFPTFPTMPTLPTLPTLAPFTFPTLPPPTT MKPLNITIDPEALMDVPEIITHWGYPVETHKVVTVDGYILTLHRILPHSKNETSKSASKTPKP VVFLQHGLLCTSSIWLLNLPRQSAGYIFADQGYDVWLGNMRGNTYSKEHTRMTSADRR FWKFSWEEMARYDLPAMINYALKTTKRQNLYYVGHSQGALTMFAKMSEDPEMSKKIRK FFAMAPVARMSHVKGLFQNLGQIYEQYNLVYQVFGDGEFLTNNIFTKLLTDIFCDQAVN NPLCENFIFAVSGPNSNQFNNSRIGIYLAHNPAGTSSRNILHFAQMVKKKRMSRFDHGK DLNLKIYGAPSPPEYDIRKINSSIYLFYSDFDWLANPKDVEGFLIPMLPSKTLKKATKLRDF NHNDFLWGMRARKEIYDKIINTIKLDQRRVKLQNSMERFFERQSRNPTSGLDEETMMRL RNETMNLD*”

Sequence ID #24. Forward DNA primer for use in amplifying and modifying the bacterial tributyrin esterase gene encoding the native bacterial esterase (SEQ ID No. 11) so as to contain part of the P12 leader, IPG976.

“CAGCTGCATACGCCATGGCAGTAATCAATATC”

Sequence ID #25. Reverse DNA primer for use in amplifying and modifying the bacterial tributyrin esterase gene encoding the native bacterial esterase (SEQ ID No. 11) so as to contain part of the P12 leader, IPG923.

“TATTAACTCAATCGTTCTTCTTGC”

Sequence ID #26. Forward DNA primer for use in amplifying and modifying the plant carboxylesterase gene encoding the native plant carboxylesterase (SEQ ID No. 14) so as to contain part of the P12 leader, IPG974.

“CAGCTGCATACGCCATGAGTATCTCCGGTGCTG”

Sequence ID #27. Reverse DNA primer for use in amplifying and modifying the plant carboxylesterase gene encoding the native plant carboxylesterase (SEQ ID No. 14) so as to contain part of the P12 leader, IPG971.

“ACTAGTTCAACCTTCGAGGCTGAG”

Sequence ID #28. Forward DNA primer for use in amplifying and modifying the plant lipase gene with SGNH motif (SEQ ID No. 17) so as to contain part of the P12 leader, IPG975.

“CAGCTGCATACGCCATGGCTTCTTCACTG”

Sequence ID #29. Reverse DNA primer for use in amplifying and modifying the plant lipase gene with SGNH motif (SEQ ID No. 17) so as to contain part of the P12 leader, IPG973.

“ACTAGTCCCTTTATGTATCCACTG”

Sequence ID #30. DNA primer used to identify nematode triglyceride lipase-cholesterol esterase gene from transgenic plants, IPG896.

“TTGCGCCACTATGGACATTG”

Sequence ID #31. DNA primer used to identify nematode triglyceride lipase-cholesterol esterase gene from transgenic plants, IPG898.

“GTGAGTGCACCTTGTGAATG”

Sequence ID #32. DNA primer used to amplify amoeba acyloxyacyl hydrolase gene from cDNA, IPG977.

“TTACTACTGCCGTAAACATTCC”

Sequence ID #33. DNA primer used to amplify amoeba acyloxyacyl hydrolase gene from cDNA, IPG978.

“TTAAACATATCCACCTTGGTTAC”

Sequence ID #34. Forward DNA primer for use in amplifying and modifying the amoeba acyloxyacyl hydrolase gene (GenBank AC117075) so as to contain part of the P12 leader, IPG979.

“cagctgcatacGCCGTAAACATTCCAG”

Sequence ID #35. Reverse DNA primer for use in amplifying and modifying the amoeba acyloxyacyl hydrolase gene (GenBank AC117075) so as to contain part of the P12 leader, IPG980.

“actagtTTAAACATATCCACCTTGGTTAC”

What is claimed is:

1. A method for selecting for resistance of a whole plant, plant tissue or plant cell to a detergent comprising introducing into the whole plant, plant tissue or plant cell an expression cassette comprising as operably linked components: 1) a promoter functional in a plant cell; 2) a nucleic acid encoding a polypeptide or protein having, at least in part, esterase activity in a plant cell; 3) a terminator functional in a plant cell, thereby obtaining a transformed whole plant, plant tissue or plant cell comprising said expression cassette; allowing the transformed whole plant, plant tissue or plant cell to express the esterase activity; and contacting the transformed whole plant, plant tissue or plant cell with the detergent thereby selecting for a whole plant, plant tissue or plant cell with resistance to the detergent.

2. The method of claim 1, wherein a secretion signal functional in the whole plant, plant tissue or plant cell is operably fused to the nucleic acid.

3. The method of claim 1, wherein the nucleic acid is a gene or gene fragment.

4. The method of claim 1, wherein the nucleic acid is a synthetic nucleic acid.

5. The method of claim 1, wherein the nucleic acid encodes a bovine pregastric esterase.

6. The method of claim 1, wherein the nucleic acid encodes a nematode lipase.

7. The method of claim 1, wherein the nucleic acid encodes a bacterial tributyrin esterase.

8. The method of claim 1, wherein the nucleic acid encodes a plant carboxylesterase.

9. The method of claim 1, wherein the nucleic acid encodes a plant lipase gene with an SGNH motif.

10. The method of claim 1, wherein the nucleic acid encodes an acyloxyacyl hydrolase.

11. The method of claim 1, wherein the nucleic acid encodes an amoeba acyloxyacyl hydrolase.

12. The method of claim 1, wherein the whole plant, plant tissue or plant cell is monocotyledonous or dicotyledonous.

13. The method of claim 1, wherein the whole plant, plant tissue or plant cell is selected from the group consisting of corn, soybean, alfalfa, sorghum, wheat, canola, tobacco, tomato, geranium (Pelargonium hortorum), rice and sugarcane.

14. A transformed whole plant, plant tissue or plant cell having resistance to a detergent and produced according to the method of claim 1, 2, 3, 4, 5, 6, 7, 8, 9, 10, 11, 12 or 13.

15. Progeny of the transformed whole plant of claim 14, whether produced sexually or asexually, wherein said progeny retains resistance to the detergent.

16. Progeny of the transformed whole plant of claim 14, whether produced sexually or asexually, wherein said progeny retains the nucleic acid and expresses esterase activity.

17. A tissue culture of the whole plant, plant tissue or plant cell of claim 14, wherein said tissue culture retains resistance to the detergent.

18. A tissue culture of the whole plant, plant tissue or plant cell of claim 14, wherein said tissue culture retains the nucleic acid and expresses esterase activity.

19. A method of plant husbandry comprising growing the whole plant of claim 14 and/or the progeny of claim 15 and 16.

20. The method of claim 1 wherein the detergent is a surfactant or a wetting agent.

21. The method of claim 1 wherein the detergent is a fatty acid ester.

22. The method of claim 1 wherein the detergent is a fatty acid ester that is formed from (i) at least one polyol chosen selected from the group consisting of polyethylene glycol comprising from 1 to 60 ethylene oxide units, sorbitan, glycerol comprising from 2 to 30 ethylene oxide units, and polyglycerols comprising from 2 to 15 glycerol units; and (ii) at least one fatty acid comprising at least one alkyl chain chosen from saturated and unsaturated, linear and branched C₈-C₂₂ alkyl chains.

23. The method of claim 1 wherein the detergent comprises mixed esters derived from (i) at least one fatty acid, at least one carboxylic acid, and glycerol; and mixed esters derived from (ii) at least one fatty alcohol, at least one carboxylic acid, and glycerol.

24. The method of claim 1 wherein the detergent comprises fatty acid esters of sugars and fatty alcohol ethers of sugars.

25. The method of claim 1 wherein the detergent is selected from the group consisting of fatty esters of glycerol, fatty esters of sorbitan, oxyethylenated fatty esters of sorbitan, ethoxylated fatty ethers, and ethoxylated fatty esters.

26. The method of claim 1 wherein the detergent is a polyoxyethylene sorbitan fatty acid ester.

27. The method of claim 1 wherein the detergent is one or more detergents selected from the group consisting of Tween 20, Span 20, Triton QS-15, Triton QS-44, PEG Monolaurate, PEG Dilaurate, PEG monostearate, PEG distearate, PEG Monooleate, or PEG Dioleate.

28. An expression cassette consisting essentially of the following operably linked components: 1) a promoter functional in a plant cell; 2) a nucleic acid encoding a polypeptide or protein having, at least in part, esterase activity in a plant cell; 3) a terminator functional in a plant cell.

29. The expression cassette of claim 28, wherein the expression cassette does not contain another selectable marker gene.

30. The expression cassette of claim 28, wherein the expression cassette does not contain a selectable marker gene selected from the group consisting of nptII (neomycin phosphotransferase) and bar.

31. A whole plant, plant tissue or plant cell comprising the expression cassette of claim 28, 29 or 30.

32. The whole plant, plant tissue or plant cell of claim 31, wherein the whole plant, plant tissue or plant cell is monocotyledonous or dicotyledonous.

33. The whole plant, plant tissue or plant cell of claim 31, wherein the whole plant, plant tissue or plant cell is selected from the group consisting of corn, soybean, alfalfa, sorghum, wheat, canola, tobacco, tomato, geranium (Pelargonium hortorum), rice and sugarcane.

34. The expression cassette of claim 28, wherein a secretion signal functional in the whole plant, plant tissue or plant cell is operably fused to the nucleic acid.

35. The expression cassette of claim 28, wherein the nucleic acid is selected from the group consisting of a gene, a gene fragment, and a synthetic nucleic acid.

36. The expression cassette of claim 28, wherein the nucleic acid codes for a bovine pregastric esterase, a nematode lipase, a bacterial tributyrin esterase, a plant carboxylesterase, a plant lipase gene with an SGNH motif, an acyloxyacyl hydrolase, and an amoeba acyloxyacyl hydrolase.

37. A recombinant DNA plant transformation vector consisting essentially of the following operably linked components: 1) a promoter functional in a plant cell; 2) a nucleic acid encoding a polypeptide or protein having, at least in part, esterase activity in a plant cell; 3) a terminator functional in a plant cell.

38. The recombinant DNA plant transformation vector of claim 37, wherein the DNA plant transformation vector does not contain another selectable marker gene.

39. The recombinant DNA plant transformation vector of claim 37, wherein the DNA plant transformation vector does not contain a selectable marker gene selected from the group consisting of nptII (neomycin phosphotransferase) and bar.

40. A whole plant, plant tissue or plant cell comprising the recombinant DNA plant transformation vector of claim 37, 38 or 39.

41. The whole plant, plant tissue or plant cell of claim 40, wherein the whole plant, plant tissue or plant cell is monocotyledonous or dicotyledonous.

42. The whole plant, plant tissue or plant cell of claim 40, wherein the whole plant, plant tissue or plant cell is selected from the group consisting of corn, soybean, alfalfa, sorghum, wheat, canola, tobacco, tomato, geranium (Pelargonium hortorum), rice and sugarcane.

43. The recombinant DNA plant transformation vector of claim 37, wherein a secretion signal functional in the whole plant, plant tissue or plant cell is operably fused to the nucleic acid.

44. The recombinant DNA plant transformation vector of claim 37, wherein the nucleic acid is selected from the group consisting of a gene, a gene fragment, and a synthetic nucleic acid.

45. The recombinant DNA plant transformation vector of claim 37, wherein the nucleic acid codes for a bovine pregastric esterase, a nematode lipase, a bacterial tributyrin esterase, a plant carboxylesterase, a plant lipase gene with an SGNH motif, an acyloxyacyl hydrolase, and an amoeba acyloxyacyl hydrolase.
